(19) United States
(12) Patent Application Publication
(10) Pub. No.: US 2024/0238419 A1
(43) Pub. Date: Jul. 18, 2024

(54) METHOD FOR CONSTITUTIVE MALT1 PROTEASE ACTIVATION

(71) Applicant: Helmholtz Zentrum München - Deutsches Forschungszentrum Für Gesundheit Und Umwelt (GmbH), Neuherberg (DE)

(72) Inventors: Daniel KRAPPMANN, München (DE); Andreas GEWIES, München (DE); Thomas J. O'NEILL, München (DE); Thomas SEEHOLZER, München (DE); Carina GRASS, München (DE)

(21) Appl. No.: 18/289,727

(22) PCT Filed: May 6, 2022
(86) PCT No.: PCT/EP2022/062243

§ 371 Date: Nov. 6, 2023

(30) Foreign Application Priority Data
May 7, 2021 (EP) 21172676.5

(51) Int. Cl. A61K 39/00; C12N 5/0783; C12N 9/64

(52) U.S. Cl. CPC A61K 39/4622; A61K 39/4611; A61K 39/4631; A61K 39/4632; A61K 39/464429; C12N 5/0636; C12N 9/6472; C12Y 304/22

(57) ABSTRACT

The present invention relates to a cell of the human immune system transduced or transfected with a T cell receptor (TCR) or chimeric antigen receptor (CAR), said cell being further modified to render its MALT1 protease activity constitutive active. The present invention further relates a said cell of the human immune system for use as a medicament. In particular, the present invention relates to said cell for use in adoptive T cell therapy. The invention also comprises a cell of the human immune system transduced or transfected with a T cell receptor (TCR) or chimeric antigen receptor (CAR), and further modified to render its MALT1 protease activity constitutive active for use in a method of treating cancer. The invention also relates to a method for generating a cell of the human immune system, comprising modifying a cell to render MALT1 protease activity constitutive active. The invention further relates to an in vitro method of enhancing the activity of a cell of the human immune system transduced or transfected with a T cell receptor (TCR) or chimeric antigen receptor (CAR), comprising modifying said cell in that MALT1 is rendered constitutive active. The invention also comprises an in vitro use of constitutive active MALT1 for enhancing the activity of a cell transduced or transfected with a T cell receptor (TCR) or chimeric antigen receptor (CAR).

### CROSS-REFERENCE TO RELATED APPLICATIONS

The present application claims the benefit of priority of European Patent Application No. 21172676.5 filed 7 May 2021, the content of which is hereby incorporated by reference in its entirety for all purposes.

### TECHNICAL FIELD OF THE INVENTION

The present invention relates to a cell of the human immune system transduced or transfected with a T cell receptor (TCR) or chimeric antigen receptor (CAR), said cell being further modified to render its MALT1 protease activity constitutive active. The present invention further relates to the use of said cell of the human immune system for adoptive T cell therapy, in particular for use in a method of treating cancer. The invention also relates to a method for generating a cell of the human immune system, comprising modifying a cell to render MALT1 protease activity constitutive active, an in vitro method of enhancing the activity of a cell of the human immune system transduced or transfected with a T cell receptor (TCR) or chimeric antigen receptor (CAR), and an in vitro use of constitutive active MALT1 for enhancing the activity of a cell transduced or transfected with a T cell receptor (TCR) or chimeric antigen receptor (CAR).

### BACKGROUND OF THE INVENTION

Immune cells, especially cytotoxic T cells, are well equipped to protect against tumor development. However, T cell populations cannot sustain their aggressive response over time. If the cells are confronted with an antigen over a longer period of time, their activity decreases. Tumor-infiltrating T cells often become immunosuppressed and fall into a kind of “state of exhaustion” in which characteristic immune checkpoint molecules (PD-1, TIGIT, LAG-3, etc.) are expressed and anti-tumor effector cytokines (IFNγ, TNFα, etc.) are downregulated, with the result that therapeutic success is suppressed or reduced. Similarly, immunosuppressive cells, such as regulatory T cells (Treg), myeloid-derived suppressor cells (MDSCs), and immunosuppressive metabolites in the tumor microenvironment can also limit therapeutic success.

Blockade of immune checkpoint molecules (inhibitors) has achieved great success in clinical trials in the treatment of various cancers (Toor S. M., Sasidharan Nair V., Elkord E. et. al. Immune checkpoint inhibitors: recent progress and potential biomarkers Experimental & Molecular Medicine 2018 volume 50, pages1-11; Sharma P., Siddiqui B. A., Anandhan S. et al., The Next Decade of Immune Checkpoint Therapy 2021 11(4): 838-857). However, only a small proportion of affected patients actually respond to these therapies, and those who initially benefit from therapy often develop resistance to therapy as it progresses. Likewise, adoptive T-cell therapy (ATC) concepts in the form of engineered T-cell receptors (TCRs) and chimeric antigen receptors (CARs), which have gained importance in recent years, have improved the treatment of tumor patients. However, even these genetically engineered T cells are often induced into a state of exhaustion.

Many concepts have been developed in the past and are currently being developed, signaling pathways investigated and molecular switches identified that could reactivate T cells from their “state of exhaustion” or prevent them from being suppressed in the first place. It is therefore an object of the present invention to provide new insights in the prevention of T cell exhaustion and to provide alternative therapeutic strategies for cancer treatment.

Therefore, the objective of the present invention is to comply with this need.

The solution of the present invention is described in the following, exemplified in the appended examples, illustrated in the figures and reflected in the claims.

### SUMMARY OF THE INVENTION

In a first aspect the present invention relates to a cell of the human immune system transduced or transfected with a T cell receptor (TCR) or chimeric antigen receptor (CAR), said cell being further modified to render its MALT1 protease activity constitutive active.

In a second aspect, the invention relates to said cell of the human immune system which is for use as a medicament.

In a third aspect said cell of the human immune system is for use in an adoptive T cell therapy.

In a fourth aspect said cell of the human immune system is for use in a method of treating cancer.

In a fifth aspect the invention relates to a method for generating a cell of the human immune system transduced or transfected with a T cell receptor (TCR) or chimeric antigen receptor (CAR), comprising modifying a cell to render MALT1 protease activity constitutive active.

In a sixth aspect, the invention covers an in vitro method of enhancing the activity of a cell of the human immune system transduced or transfected with a T cell receptor (TCR) or chimeric antigen receptor (CAR), comprising modifying said cell in that MALT1 is rendered constitutive active.

Finally, in a seventh aspect the present invention envisages the in vitro use of constitutive active MALT1 for enhancing the activity of a cell transduced or transfected with a T cell receptor (TCR) or chimeric antigen receptor (CAR).

### BRIEF DESCRIPTION OF THE FIGURES

FIG. 1: Combination of tumor antigen recognition and MALT1 protease activation for boosting adoptive T cell therapy. After collecting and isolating T cells from a subject, said cells are subjected to transfection or viral transfer of a TCR or CAR, in addition to gene editing to render MALT1 protease constitutively active. The obtained tumor-reactive T cells are expanded and subsequently transferred back to the subject.

FIG. 2: MALT1 protease activation upon loss of TRAF6 binding in human Jurkat T cells. (A) scheme of MALT1A and MALT1B protein domains. (B) Western Blot (WB) analysis showing detection of active MALT1 in untreated reconstituted MALT1 KO Jurkat T cells by labeling with bio-MALT1 ABP and substrate cleavage. Quantification was done by determining the ratio of active (pulldown) to total (lysate) MALT1 from three independent experiments. Active MALT1 and cleavage of substrates was detected in resting Jurkat T cells containing the TRAF6 binding mutants MALT1A E316A/E806A or MALT1B E795A, and constitutive protease activity was abolished in MALT1B active site mutant C453A. (C) WB analysis showing MALT1 KO Jurkat T cells reconstituted with human MALT1B WT or T6BM mutants MALT1B E795A and MALT1B E795D, the cells were left untreated or stimulated with P/I (30 min) before assessment of MALT1 substrate cleavage.

FIG. 3: MALT1 protease activation in TRAF6 deficient human Jurkat T cells. (A) WB analysis showing detection of active MALT1 in wt Jurkat T cells and TRAF6 KO Jurkat T cells by labeling with bio-MALT1 ABP and pulldown before WB. Quantification was done by determining the ratio of active (pulldown) to total (lysate) MALT1 from three independent experiments. (B) WB analysis showing MALT1 substrate cleavage in TRAF6 KO Jurkat T cells reconstituted with mock, TRAF6 WT or mutant C70A (E3 ligase mutant) with and without P/I stimulation. (C) WB analysis showing MALT1 substrate cleavage in TRAF6 KO Jurkat T cells reconstituted with TRAF6 WT or oligomerization mutant R88A/F118A with and without P/I stimulation.

FIG. 4: MALT1 TBM mutation or TRAF6 deficiency in murine T cells renders MALT1 protease constitutively active. (A) and (B) WB analysis showing constitutive cleavage of MALT1 substrate CYLD, HOIL-1, Regnase-1, Roquin-1/2 in isolated splenic CD4+ T cells from three control, five Malt1 TBM-T (A) and five Traf6-ΔT (B) mice. (C) Constitutive MALT1 activity activates T cells leading to a strong increase in the population of CD4 and CD8 T effector memory (EM) cells. (D) and (E) Expression of I_{K}BNS and ICOS in CD4+ and CD8+ T cells from spleen of control, Malt1 TBM-T and Traf6-ΔT mice. Constitutive MALT1 protease activation induces the expression of T cell activator genes controlled by MALT1 protease in CD4 and CD8 T cells such as I_{K}BNS (D) or ICOS (E).

FIG. 5: MALT1 protease activation and T cell activation induced by loss of TRAF6 can be reverted by MALT1 inhibitor treatment. (A) Schematic representation of MALT1 inhibitor (MLT-985) treatment schedule of Traf6-ΔT mice starting 8 weeks after birth. (B) WB analysis showing MALT1 substrate cleavage in isolated splenic CD4+ T cells from Traf6-ΔT mice treated with vehicle (3) or MLT-985 (4). Asterisk indicates unspecific band. (C) Relative numbers of T_{EM} CD4+ and CD8+ T cells in spleen of vehicle or MLT-985 treated Traf6-ΔT mice or Wt mice (untreated). (D) Expression of I_{K}BNS and ICOS in CD4+ T cells from spleen of vehicle or MLT-985 treated Traf6-ΔT mice or Wt mice (untreated).

FIG. 6: Constitutive MALT1 protease activity drives lethal inflammation in Malt1 TBM mice. (A) Scheme of Glu (E) to Ala (A) exchanges in the two T6BMs of MALT1A in Malt1 TBM mice (top) and additional Cys (C) to Ala (A) exchange in Malt1 TBMPM mice (bottom). (B) Kaplan-Meier-Curve showing the survival of Malt1^{TBMPM/TBMPM} (n=13), Malt 1^{TBMPM/+}(n=13), and Malt 1^{TBM/TBM} mice (n=10). One Malt1^{TBMPM/TBMPM} mouse died at day 27 of unknown cause. (C) WB analysis showing biochemical analyses of MALT1 substrate cleavage (WB) in isolated splenic CD4+ T cell (unstimulated and P/I stimulation) from Malt 1^{TBMPM/+} and Malt 1^{TBMPM/TBMPM} mice. (D) Relative numbers of CD44hiCD62L′ CD4+ and CD8+ effector/memory T (T_{EM}) cells in SPL and LN of Malt 1TBM+ and Malt 1^{TBM/TBM} mice. (E) Relative numbers of CD44 CD62L″ CD4+ and CD8+ T_{EM} cells in SPL and LN of Malt 1^{TBMPM/+} and Malt 1^{TBMPM/TBMPM} mice. (F) Relative numbers of CD4+ FoxP3+ Treg cells in SPL of Malt 1 TBM/+ and Malt1 mice. (G) Relative numbers of CD4+ FoxP3+ Treg cells in SPL of Malt 1^{TBMPM/+} and Malt 1^{TBMPM/TBMPM} mice. (H) Relative numbers of effector (e)Treg cells (CD4+CD44hCD62L^{lo}) in SPL of Malt1TBM+ and Malt1^{TBM/TBM} mice. (I) Concentration of indicated inflammatory cytokines in the sera of Malt1^{TBM/+} and Malt1^{TBMTBM} mice. (J) Concentration of indicated inflammatory cytokines in the sera of Malt1TBMPM+ and Malt 1^{TBMPM/TBMPM} mice.

FIG. 7: Loss of TRAF6 binding solely in T6BM2 is sufficient for constitutive MALT1 protease activity and lethal inflammation in Malt1 TBM2 mice. (A) Scheme of Glu (E) to Asp (D) exchange in T6BM2 of MALT1A in Malt1 TBM2 mice. (B) Relative numbers of CD44^{hi}CD62L^{lo} CD4+ and CD8+ effector/memory T (T_{EM}) cells in SPL and LN of Malt1TBM2/+ and Malt 1TBM2/TBM2 2 mice.

FIG. 8: OT1+ T cells containing Malt1 TBM mutations have improved tumor targeting and show enrichment within the tumor microenvironment (A) CD45.2 donor-derived OT1+ CD8+ T cells with or without Malt1 TBM mutations were purified and transferred to CD45.1 receptor mice that were injected subcutaneously with B16-Ova tumor cells. (B) Relative numbers of CD45.2 donor-derived OT1+ T cells, which specifically recognize the Ova antigen on the B16-Ova tumor cells, where determined in the tumor tissue, as well as in spleen, and axillary lymph nodes, of CD45.1-expressing recipient mice measured 3 weeks after tumor injection. (C) The augmented recruitment of cancer cell-recognizing OT1+ T cells into the tumor tissue by the MALT1 activating mutations provides a distinct advantage for the anti-tumor response, which will boost the cytotoxic T cell's attack against the cancer cells, for instance after injection of innate immune stimulating agents (such as CpG oligodeoxynucleotides; CpG ODN) or the SIINFEKL (Ova) peptide. Thus, by improved recruitment of tumor recognizing T cells, constitutive MALT1 protease activation will generate an improved anti-tumor response.

### DETAILED DESCRIPTION OF THE INVENTION

If “exhausted” cytotoxic T cells could be re-armed, this could significantly improve the body's fight against chronic infections and tumors. The inventors developed the concept in this invention via modification of the MALT1 (“Mucosa-Associated Lymphoid Tissue Lymphoma Translocation Protein 1”) protease. MALT1 regulates immune cell function through its adaptor function which triggers NF-_{K}B signaling and its proteolytic function which augments immune signaling and responses.

MALT1 is activated in T cells upon binding of a TCR to a tumor ligand (antigen) and promotes T cell development and effector functions. At the same time, MALT1 has a central role in the suppressive function of Treg cells. In the current work, it has now been found that systemic expression of constitutively active MALT1 mutant in mice induces strong autoimmunity and inflammation. The “missense” mutations in the TRAF6 binding motifs of the Malt1 gene inserted by CRISPR/Cas9 resulted in inhibition of TRAF6 binding to MALT1, rendering MALT1 protease constitutively active and thereby inducing impaired T cell homeostasis (immune system balance) triggered by a strong increase in effector and effector memory T cells. Importantly, these effector and effector memory T cells are not suppressed by regulatory T cells.

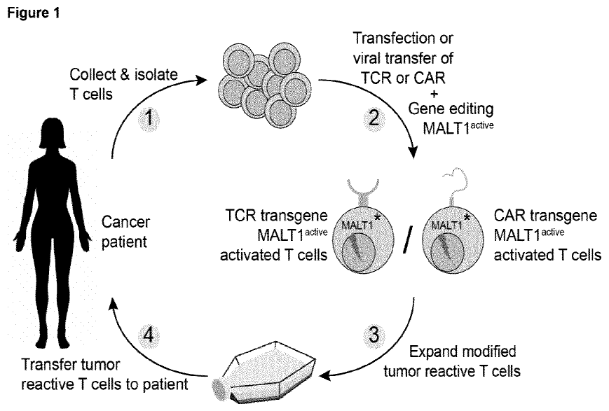

In accordance with the present invention, it is foreseen that these “missense” mutations in the Malt1 gene are only inserted into the TCR- or CAR-edited T cells used for adoptive cell therapy to achieve an improved anti-tumor response compared to T cells with the respective wild type Malt1 gene, while avoiding any systemic activation of MALT1 with possible corresponding negative consequences. Therefore, the mutations result in MALT1 being constitutively active only in these tumor-reactive T cells. In this context, by “improved anti-tumor response” it is meant that the MALT1-mutated T cells display higher potential in impairing tumor growth compared to wild type T cells. Impaired tumor growth can be caused either by the higher cytotoxic capacity of the MALT1-mutated T cells, e.g. by increased production of inflammatory cytokines such as INFγ, TNFα etc., or by the prevention of a “state of exhaustion” characterized by expression of immune checkpoint molecules, such as PD-1, TIGIT, LAG-3, etc. mentioned elsewhere herein. This has the consequence that these tumor-specific MALT1-TCR/CAR-edited T cells induce a more effective and long lasting anti-tumor immune response due to permanently activated MALT1. The effector T cells thus remain more active and are not suppressed by regulatory T cells, and MALT1 does not have to be activated first by antigen receptor stimulation (TCR/CAR-tumor antigen) and the T cells do not reach a state of “exhaustion”. A scheme to apply the innovative approach according to the present invention is shown in FIG. 1.

Accordingly, the present invention relates to a cell of the human immune system transduced or transfected with a T cell receptor (TCR) or chimeric antigen receptor (CAR), which is further modified to render its MALT1 protease activity constitutive active.

In such engineered T cell therapy (CAR-T cell therapy and T cell receptor (TCR)-T cell therapy), sufficient amounts of blood are drawn from patients to obtain enough peripheral blood mononuclear cells (PBMCs) for engineered T cell manufacturing. The T cells are purified from patient PBMCs. After activation and amplification in vitro, T cells are modified by viral vector transfection, such as lentivirus transfection or retrovirus transfection, to express specific CARs/TCRs on the T cell surface. Following amplification and quality control, CAR-T cells/TCR-T cells are infused into the patient body to improve antitumor ability. In particular, CAR-T cell therapy is a revolutionary targeted immunotherapy. Chimeric antigen receptor (CAR) T cells (or CAR T cells) are genetically modified and express synthetic receptors with specificities against tumor-antigens. A CAR protein comprises a single-chain variable fragment (scFv) with binding capacity for one specific tumor associated antigen, linked via a transmembrane peptide to intracellular co-stimulatory domains such as CD28, OX40 and CD137. These peptides are subsequently joined to the signaling domains of the TCRζ chain that activates the CAR T cell, if it binds its epitope on a tumor cell. Subsequent release of granzymes and perforins leads to tumor cell lysis (June C H, O'Connor R S, Kawalekar O U, Ghassemi S, Milone M C. CAR T cell immunotherapy for human cancer. Science (New York, N.Y.) 2018; 359:1361-65). These synthetic receptor molecules enable an MHC-independent T cell activation unlike the common reaction via the TCR complex. (Gideon Gross, Tova Waks, and Zelig Eshhar. Expression of immunoglobulin-T-cell receptor chimeric molecules as functional receptors with antibody-type specificity. Proc. Natl. Acad. Sci. USA 1989:10024-28; Kuwana Yea. Expression of chimeric receptor composed of immunoglobulin-derived V regions and T-cell receptor-derived C regions. Biochemical and biophysical research communications 1987:960-68). This represents an important benefit in tumor cell recognition since the loss of MHC-associated antigen presentation is a major immune escape strategy by malignant cells (Garrido F, Aptsiauri N, Doorduijn E M, Garcia Lora A M, van Hall T. The urgent need to recover MHC class I in cancers for effective immunotherapy. Current opinion in immunology 2016; 39:44-51).

In particular, according to the present invention the cell of the human immune system which is transduced or transfected with a TCR or a CAR, and being further modified to render its MALT1 protease activity constitutive active is a T cell, NK-cell, NKT-cell, a B cell or a macrophage. Preferably, the cell of the human immune system is a CD8+ T cell, a CD4+ T cell or a Treg cell.

The protein MALT1 (PCASP1) is part of the paracaspase family and shows proteolytic activity upon stimulation, such as binding of a foreign antigen presented by antigen presenting cells (APC) to the cognate TCR expressed on the surface of the antigen-specific T cells. Such stimulation is often, but not always, accompanied by a second co-stimulus, such as the CD28 receptor on the T cell binding to the CD80 (B7.1) or CD86 (B7.2) proteins on the surface of the APC. In contrast, tonic TCR signaling, defined by low affinity binding of the TCR to a self-reactive peptide antigen, which can be presented on the surface of any cell by MHC molecule and in the absence of a co-stimulus, is not able to activate MALT1 protease. In an adaptive immune response, MALT1 paracaspase exerts a non-catalytic and catalytic function within the CARD11-BCL10-MALT1 (CBM) signaling complex upon T-cell stimulation (Juilland, M. & Thome, M. Holding All the CARDs: How MALT1 Controls CARMA/CARD264 Dependent Signaling. Frontiers in immunology 9, 1927, (2018); Ruland, J. & Hartjes, L. CARD-BCL-10-MALT1 signalling in protective and pathological immunity. Nature reviews. Immunology, (2018)). MALT1 scaffolding is required to recruit the E3 ligase TRAF6 to the CBM complex, which triggers activation of canonical NF-_{K}B signaling (Meininger, I. et al. Alternative splicing of MALT1 controls signalling and activation of CD4(+) T cells. Nat Commun 7, 11292, doi: 10.1038/ncomms11292 (2016); Oeckinghaus, A. et al. Malt1 ubiquitination triggers NF-kappaB signaling upon T-cell activation. EMBO J 26, 4634-4645, doi: 10.1038/sj.emboj.7601897 (2007); Sun, L., Deng, L., Ea, C. K., Xia, Z. P. & Chen, Z. J. The TRAF6 ubiquitin ligase and TAK1 kinase mediate IKK activation by BCL10 and MALT1 in T lymphocytes. Mol Cell 14, 289-301, doi: 10.1016/s1097-2765(04)00236-9 (2004)). In parallel, MALT1 protease activity catalyzes the cleavage of substrates involved in signaling (such as A20/TNFAIP3, BCL10, CYLD, HOIL-1/RBCK1 and MALT1) (Klein, T. et al. The paracaspase MALT1 cleaves HOIL1 reducing linear ubiquitination by LUBAC to dampen lymphocyte NF-kappaB signalling. Nature communications 6, 8777, doi: 10.1038/ncomms9777 (2015); Staal, J. et al. T-cell receptor-induced JNK activation requires proteolytic inactivation of CYLD by MALT1. The EMBO journal 30, 1742-1752, doi:10.1038/emboj.2011.85 (2011); Rebeaud, F. et al. The proteolytic activity of the paracaspase MALT1 is key in T cell activation. Nature immunology 9, 272-281, doi:10.1038/ni1568 (2008); Coornaert, B. et al. T cell antigen receptor stimulation induces MALT1 paracaspase-mediated cleavage of the NF-kappaB inhibitor A20. Nature immunology 9, 263-271,doi:10.1038/ni1561 (2008)), transcription (RelB) (Hailfinger, S. et al. Malt1-dependent RelB cleavage promotes 285 canonical NF-kappaB activation in lymphocytes and lymphoma cell lines. Proceedings of the National Academy of Sciences of the United States of America 108, 14596-14601, doi:10.1073/pnas. 1105020108 (2011)) and RNA metabolism (Roquin-1/2, Regnase-1 and N4BP1) (Jeltsch, K. M. et al. Cleavage of roquin and regnase-1 by the paracaspase MALT1 releases their cooperatively repressed targets to promote T(H) 17 differentiation. Nat Immunol 15, 1079-1089, doi: 10.1038/ni.3008 (2014); 14 Uehata, T. et al. Malt1-induced cleavage of regnase-1 in CD4(+) helper T cells regulates immune activation. Cell 153, 1036-1049, doi:10.1016/j.cell.2013.04.034 (2013); Yamasoba et al., N4BP1 restricts HIV-1 and its inactivation by MALT1 promotes viral reactivation. Nat. Microbiol., 2019 Sep;4(9):1532-1544. doi: 10.1038/s41564-019-0460-3.). MALT1 paracaspase mutant (PM) mice revealed that protease activity not only controls activation of conventional T (Tconv) cells, but also development and function of regulatory T (Treg) cells (Bornancin, F. et al. Deficiency of MALT1 paracaspase activity results in unbalanced Regulatory and effector T and B cell responses leading to multiorgan inflammation. J Immuno/194, 3723-3734, doi: 10.4049/jimmunol. 1402254 (2015); Demeyer, A. et al. MALT1 Proteolytic Activity Suppresses Autoimmunity in a T Cell Intrinsic Manner. Frontiers in immunology 10, 1898, doi: 10.3389/fimmu.2019.01898 (2019); Gewies, A. et al. Uncoupling Malt1 threshold function from paracaspase activity results in destructive autoimmune inflammation. Cell Rep 9, 1292-1305, doi: 10.1016/j.celrep.2014.10.044 (2014)). T-cell-specific ablation of TRAF6 in mice was reported to be dispensable for NF-_{K}B activation, but disrupted immune homeostasis leading to autoimmune inflammation (King, C. G. et al. TRAF6 is a T cell-intrinsic negative regulator required for the maintenance of immune homeostasis. Nat Med 12, 1088-1092, doi: 10.1038/nm1449 (2006). In vitro experiments showed that destruction of TRAF6 binding motifs (T6BMs) in MALT1 abolished NF-_{K}B signaling in T-cells (Meininger, I. et al. Alternative splicing of MALT1 controls signalling and activation of CD4(+) T cells. Nat Commun 7, 11292, doi:10.1038/ncomms11292 (2016)). Two conserved splice variants of MALT1 exist: MALT1A and MALT1B.

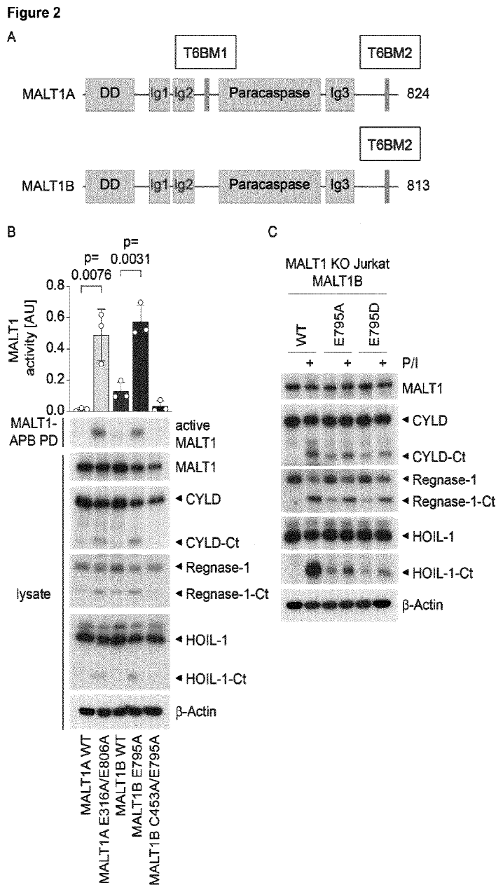

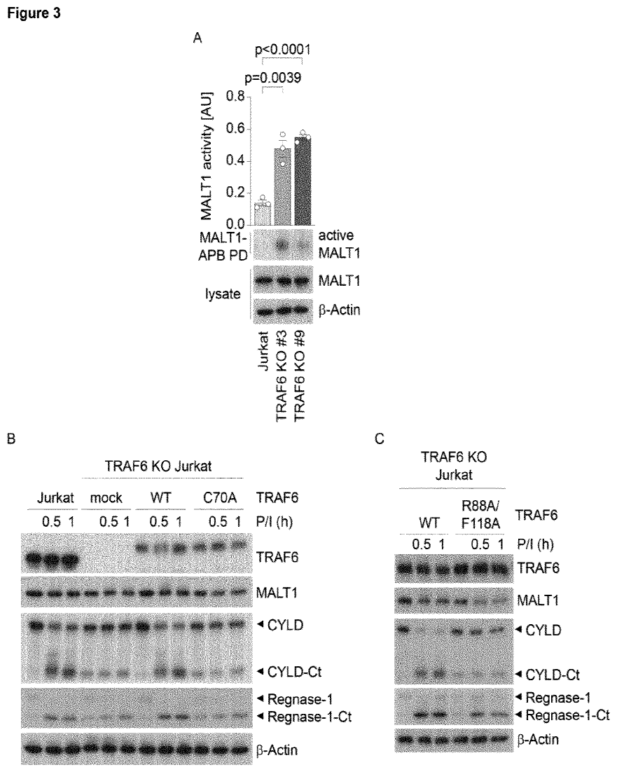

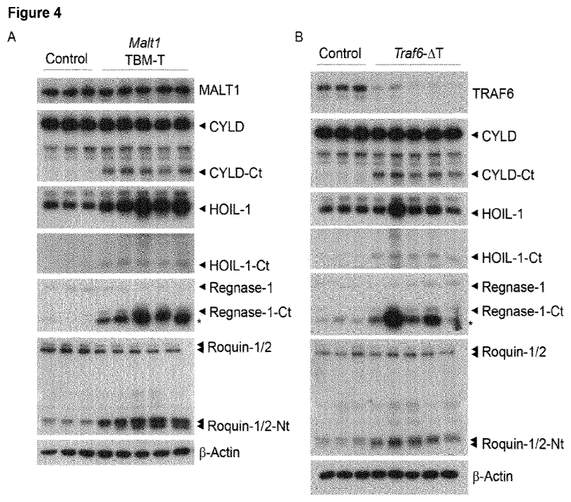

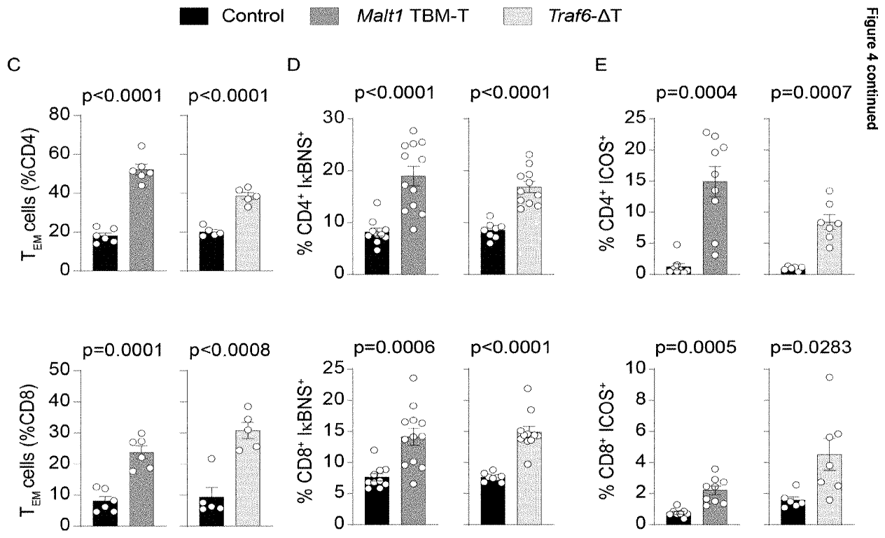

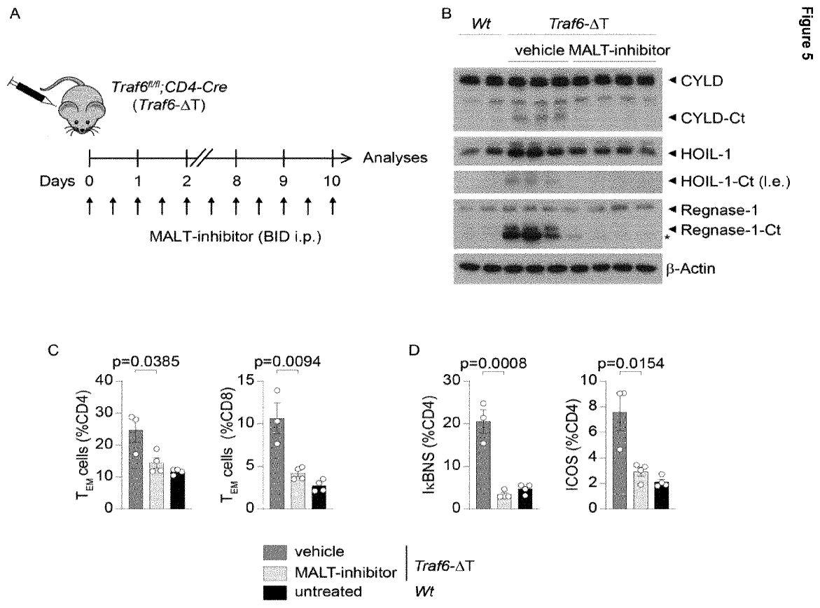

As it is known to the skilled person, the term “constitutive” as used in biotechnology, refers to a spontaneous and sustained activation of a protein or gene or an enzymatic activity. For example, a protein receptor is considered constitutively active when it is able to transduce a signal even in absence of its specific ligand which triggers the signal transduction in normal, i.e. physiological conditions. Similarly, a constitutively active protease may be a protease which is able to bind and degrade its substrates even in absence of an activating stimulus, for example the binding of an activator protein, or by removing a negative regulator for example binding of a suppressor protein, or by an expression of a dominant activator, for example by a gain-of-function mutation leading to activation. In the context of the present invention, a MALT1 constitutive active protease is a MALT1 protease which is able to degrade its substrates, defined elsewhere herein, in absence of a stimulus or the removal of a negative regulator or expression of a dominant activator. In this context, the stimulation or stimulus may refer to “stimulation of a cell of the immune system”, as defined elsewhere herein, which refers to activation of intracellular signalling pathways, for example by ligand-receptor interactions, resulting in activation of a cell of the immune system. Stimulation leading to activation of a cell of the immune system is further defined elsewhere herein. In particular, as used herein the MALT1 protease activity of the cell of the immune system according to the invention is constitutive—meaning the protease activity (i.e. degradation of MALT1 substrates) is de-repressed and thereby increased—even in absence of strong cognate antigenic stimulation of said cell. In particular, according to the present invention, the constitutive active MALT1 protease activity of the cell of the human immune system according to the invention is characterized by an increased MALT1 protease activity in comparison to MALT1 protease activity of a cell of the immune system transduced or transfected with a T cell receptor (TCR) or chimeric antigen receptor (CAR), but which is not further modified to render its MALT1 protease activity constitutive active. By increased protease activity it is meant that the amount of cleaved MALT1 substrates is increased compared to the amount of cleaved MALT1 substrates in a cell of the immune system transduced or transfected with a T cell receptor (TCR) or chimeric antigen receptor (CAR), but which is not further modified to render its MALT1 protease activity constitutive active. The amount of cleaved MALT1 substrates can be quantified by means and methods known to the skilled artisan. This includes Western Blots showing in a qualitative or quantitative manner for example by densitometric analysis of intensities of full length and cleavage bands for the substrates. MALT1 protease detection is also achieved by measuring MALT1 protease activity using biotin or fluorophore-labelled MALT1 activity based probes and subsequent quantification of total versus active MALT1 for example by densitometric or fluorogenic analysis (see FIG. 2B and FIG. 3A). Constitutive MALT1 protease activity can also be detected and quantified by differences in the expression of downstream targets of the MALT1 protease, for example induction of NFKBIZ/I_{K}Bζ, NFKBID/I_{K}BNS or ICOS mRNAs using quantitative polymerase chain reaction (qPCR) or protein by flow cytometry (FIGS. 4D and 4E; FIG. 5D).

According to the invention, the MALT1 protease activity is determined by cleavage of MALT1 substrates, such as Regnase-1, Roquin1, Roquin2, N4BP1, HOIL-1/RBCK1, CYLD, A20/TNFAIP3, BCL10, MALT1 or RelB. In a preferred embodiment of the invention, the MALT1 protease activity is determined by cellular activity-based assay utilizing a MALT1 activity-based probe (ABP) such as Biotin-LVSR-AOMK, Biotin-LRSR-AOMK or BODIPY-LVPipR-AOMK (A. C. Eitelhuber et al., Activity-based probes for detection of active MALT1 paracaspase in immune cells and lymphomas. Chemistry & biology 22, 129-138 (2015); M. A. T. van de Plassche et al., Use of Non-Natural Amino Acids for the Design and Synthesis of a Selective, Cell-Permeable MALT1 Activity-Based Probe. J Med Chem 63, 3996-4004 (2020)). FIG. 2B and FIG. 3A shows an example of such a cellular activity-based assay. Preferred cellular activity-based assay are described in Example 1.

Preferably, the MALT1 protease activity is rendered constitutive active by abolishing MALT1 interaction with TRAF6. The E3 ligase TRAF6 acts as positive and negative regulator in immune signaling. In T-cells, ablation of TRAF6 causes spontaneous multi-organ inflammation. The inventors generated Jurkat T cells with mutations that disrupt the two functional TRAF6 binding motifs (T6BMs) of the Malt1 gene (FIG. 2A). Two conserved splice variants of MALT1 exist. While the C-terminal T6BM2 is present in MALT1A (aa 804-809) and MALT1B (aa 793-798), the T6BM1 within the alternatively spliced exon 7 is only encoded in MALT1A (aa 314-319). Combined mutation of both T6BMs in MALT1 prevents TRAF6 association and abolishes TCR/CD28 induced NF-_{K}B activation.

The inventors show that loss of interaction with TRAF6 causes a spontaneous MALT1 protease activation, i.e MALT1 becomes constitutively active, as defined elsewhere herein. In particular “constitutive active” MALT1 as used herein means that, upon loss of interaction with TRAF6, the protease has an increased protease activity (see FIG. 2B) and that this increased protease activity is also observed in absence of cognate antigen-triggered T cell stimulation (see FIG. 2C). “T-cell stimulation” as used herein refers to activation of intracellular signaling pathways, for example by antigenic ligand-receptor interactions, resulting in T-cell activation, in particular, in this context T cell stimulation may refer to a TCR ligation by a cognate antigen, which is required to induce an effective immune response, i.e. as in the case of an immune response triggered by an infection or a tumor-specific antigen. In contrast, tonic T cell stimulation is triggered by interaction with self-peptide-loaded MHC molecules, which leads to a low level of T cell activation, which does not lead to an effective immune response under physiological conditions. Tonic T cell stimulation alone is also not able to significantly increase MALT1 protease activity as determined by substrate cleavage, activity-based assays or induction of downstream target. As is known to the skilled in the art, small chemical compounds can be used to stimulate T-cells and cytokine production. For example, T-cell stimulation can be achieved by use of lonomycin. lonomycin is an ionophore produced by the bacterium Streptomyces conglobatus. It is used to stimulate the intracellular production of the cytokines, interferon, perforin, IL-2, and IL-4 usually in conjunction with PMA. PMA (phorbol-12-myristate-13-acetate) is an activator of protein kinase C (PKC) and a known tumor promoter. As used herein, the conjunct use of PMA and lonomycin to stimulate T-cell activation is referred to as “P/I”. P/I stimulation bypasses the T-cell membrane receptor complex and leads to activation of several intracellular signaling pathways, resulting in T-cell activation and production of a variety of cytokines. The constitutive, increased activity of MALT1 results in an augmented cleavage of MALT1 substrates such as CYLD, Regnase-1, HOIL-1, and Roquin 1/2. In particular, as defined elsewhere herein, in the context of the present invention the constitutive active MALT1 protease activity of the immune cell according to the invention refers to an increased MALT1 protease activity in comparison to MALT1 protease activity of a cell of the immune system transduced or transfected with a T cell receptor (TCR) or chimeric antigen receptor (CAR), but which is not further modified to render its MALT1 protease activity constitutive active. In this respect, by “increased” MALT1 protease activity it is meant that the constitutive active MALT1 protease in said immune cell shows an augmented cleavage of MALT1 substrates when compared to a MALT1 protease in an immune cell which is transduced or transfected with a T cell receptor (TCR) or chimeric antigen receptor (CAR), but which is not further modified to render its MALT1 protease activity constitutive active. Preferably, said substrates are selected from a group comprising CYLD, Regnase-1, HOIL-1, and Roquin 1/2.

Preferably, the MALT1 protease activity is increased, as defined herein by an increase of substrate cleavage in Western Blot, direct protease activity as detected by activity-based probes or the expression of downstream targets in using qPCR or flow cytometry. Based on quantification of activity-based probes the inventors see that the activity of MALT1 protease activity is increased by about 200-5000% in MALT1 TRAF6 binding mutants or after loss of TRAF6 when compared to unmanipulated controls (FIG. 2B and FIG. 3A). Flow cytometry of MALT1 protease downstream targets I_{K}BNS and ICOS revealed a 200-2000% increase in expression after MALT1 TBM mutation or TRAF6 loss in primary CD4 T cells.

According to the invention, MALT1 interaction with TRAF6 is abolished by inactivating the TRAF6 binding motif 1 (T6BM1) of human MALT1A and/or the TRAF6 binding motif 2 (T6BM2) of human MALT1A or human MALT1B (see FIG. 2A for a scheme of MALT1 protease domains). In particular, the T6BM1 encompasses amino acids 314 to 319 of human MALT1A shown in SEQ ID NO: 1 and T6BM2 may encompass amino acids 804 to 809 of human MALT1A shown in SEQ ID NO: 1 or amino acids 793 to 798 of human MALT1B shown in SEQ ID NO: 2. Generally, the term “inactivation” may refer to introduction of mutations leading to amino acid substitutions which cause a loss of function of the domain of a protein/protein interaction in question. In the context of the present invention, “inactivating” the TRAF6 binding motifs refers to the introduction of mutations in the Malt1 gene encoding MALT1A and/or the Malt1 gene encoding MALT1B, resulting in amino-acid substitution in TRAF6 binding motif 1 (T6BM1) of human MALT1A and/or TRAF6 binding motif 2 (T6BM2) of human MALT1A or human MALT1B, wherein said mutations inactivate the ability of said motifs to bind and consequently interact with TRAF6, thereby impeding the MALT1-TRAF6 interaction. The introduction of mutations in a specific gene, here MALT1, is also known as mutagenesis. The term “mutagenesis” as used herein means that the experimental conditions are chosen such that the amino acid naturally occurring at a given sequence position of the Malt1 gene can be substituted by at least one amino acid that is not present at this specific position in the respective natural polypeptide sequence. The term “mutagenesis” may also comprise the (additional) modification of the length of sequence segments by deletion or insertion of one or more amino acids. Such an insertion or deletion may be introduced independently from each other in any of the peptide segments that can be subjected to mutagenesis in the disclosure.

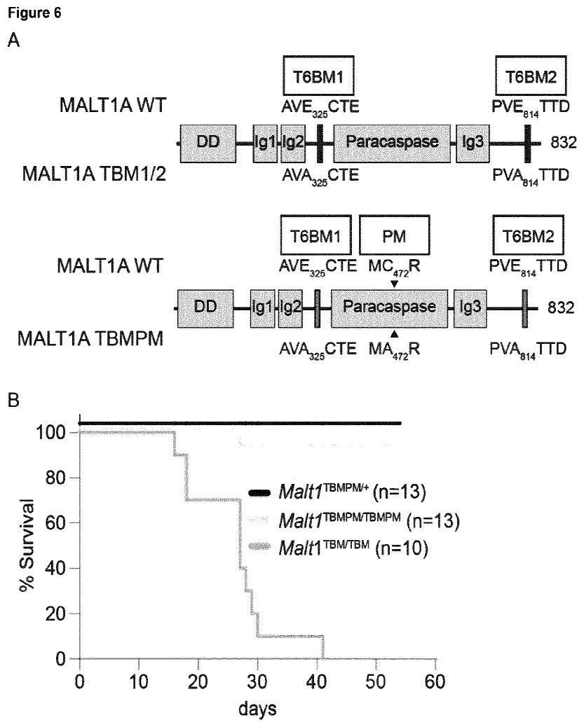

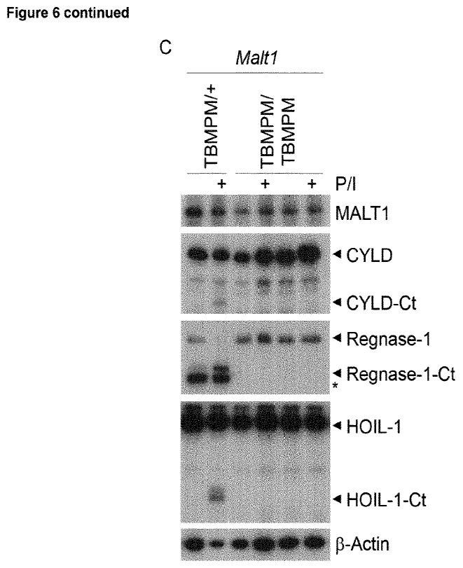

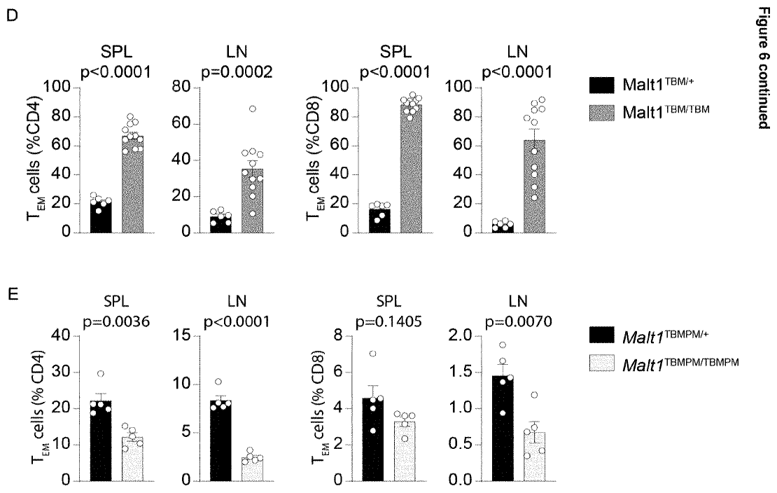

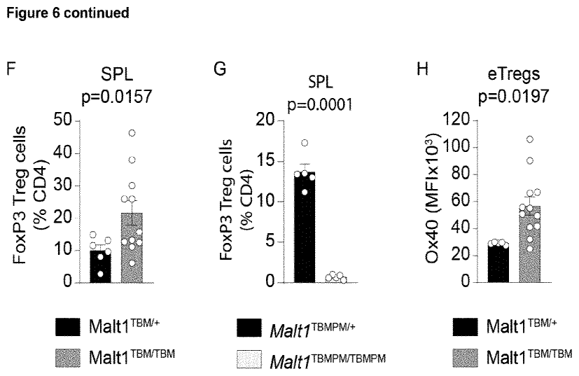

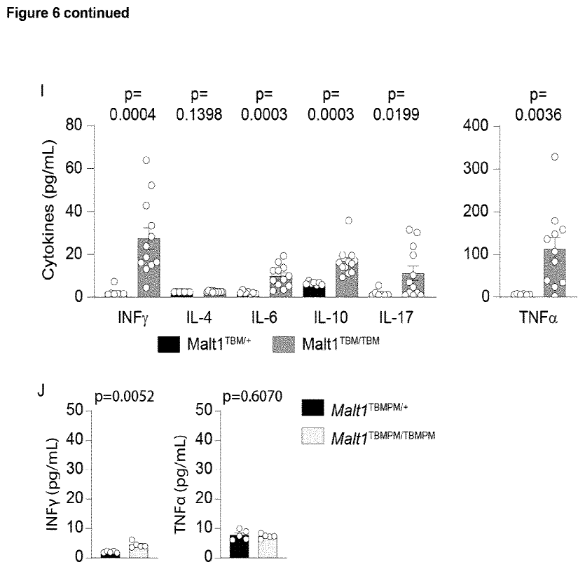

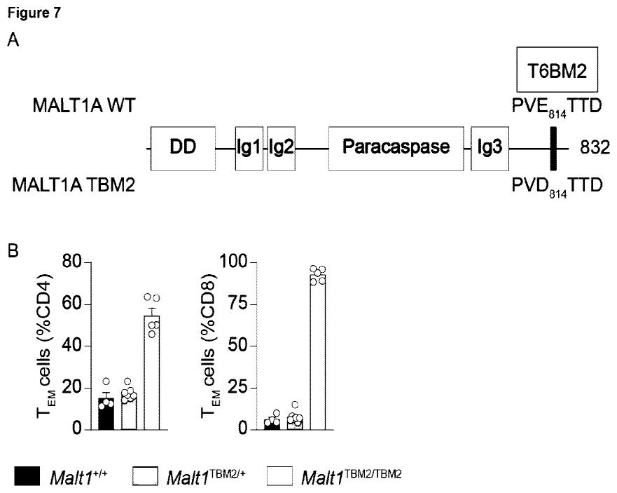

Mutations in the Malt1 gene may be introduced by techniques of gene editing known to the skilled artisan, which are defined elsewhere herein. In preferred embodiments, missense mutations in the Malt1 gene may be introduced by CRISPR/Cas9 editing, for example, in embryonic stem (ES) cells yielding a MALT1-TRAF6 binding-deficient mutant (TBM) (FIG. 6) or directly in zygotes yielding the MALT1-TRAF6 binding-deficient mutant (TBM2) (FIG. 7). Importantly, upon loss of TRAF6 or MALT1-TRAF6 binding, the inventors surprisingly found that MALT1 protease is rendered constitutively active. As used herein, a MALT1 mutant deficient in the binding to TRAF6 because of mutations in the T6BM domains may also be referred to as Malt1-TBM (see for example FIG. 4A). In a preferred embodiment of the invention, the MALT1 protease activity is rendered constitutive active by substituting E for another amino acid, preferably E→A or E→D at a position in a MALT1A protein corresponding to position 316 in SEQ ID NO: 1. In a further preferred embodiment, the MALT1 protease activity is rendered constitutive active by substituting E for another amino acid, preferably E→A or E→D at a position in a MALT1A protein corresponding to position 806 of the MALT1A protein shown in SEQ ID NO: 1, or at a position in a MALT1B protein corresponding to position to position 795 of the MALT1B protein shown in SEQ ID NO: 2. Preferably, MALT1 protease activity is rendered constitutive active by (i) substituting Y→A at a position in a MALT1A protein corresponding to position 657 of the MALT1A protein shown in SEQ ID NO: 1, (ii) substituting an amino acid at a position in a MALT1A protein corresponding to position 506 of the MALT1A protein shown in SEQ ID NO: 1, wherein the amino acid substitution is selected from the group consisting of L→A, L→G, and L→K, (iii) substituting N→A at a position in a MALT1A protein corresponding to position N508 of the MALT1A protein shown in SEQ ID NO: 1, or (iv) substituting Y→A at a position in a MALT1A protein corresponding to position Y367 of the MALT1A protein shown in SEQ ID NO: 1. In another preferred embodiment, MALT1 protease activity is rendered constitutive active by (i) substituting Y→A at a position in a MALT1B protein corresponding to position 646 in MALT1B shown in SEQ ID NO:2, (ii) substituting Y→A at a position in a MALT1B protein corresponding to position L495 of the MALT1B protein shown in SEQ ID NO:2, (iii) substituting Y→A at a position in a MALT1B protein corresponding to position N495 of the MALT1B protein shown in SEQ ID NO:2, or (iv) substituting Y→A at a position in a MALT1B protein corresponding to position Y356 of the MALT1B protein shown in SEQ ID NO:2. Said substitutions in the TRAF6 binding motifs of MALT1 lead to a constitutive MALT1 protease activation, as defined elsewhere herein, meaning that, upon loss of interaction with TRAF6, the protease MALT1 is rendered constitutively active (see FIG. 2B and FIG. 4A) and that this increased protease activity is also observed in absence of additional T cell stimulation (meaning additional TCR ligation by a cognate antigen which is required to induce an effective immune response) (see FIG. 2C).

In some embodiments, the MALT1 protease activity is rendered constitutive active by inactivating TRAF6 through a) inactivating TRAF6 gene expression, b) rendering TRAF6 E3 ligase inactive or c) abolishing TRAF6 oligomerization. With regard to inactivating TRAF6 gene expression, methods of gene inactivation are known to the skilled in the art. Gene inactivation can for example be achieved by gene knock out, in which one of an organism's genes is made inoperative (“knocked out” of the organism). Gene knock-out can be achieved in different ways, for example Homologous recombination or site-specific nucleases (such as Zinc-fingers, TALENS, CRISPR/Cas9 and meganucleases). Alternatively, gene inactivation can be achieved by RNA interference. In preferred embodiments of the invention, the inactivation of the TRAF6 gene expression is achieved by CRISPR/Cas9. (See FIG. 3 and FIG. 4B). The inventors demonstrate herein that inactivation of TRAF6 gene (here, TRAF6 KO, achieved for example via CRISPR/Cas9) induces constitutive MALT1 protease activation in Jurkat T-cells, as shown for example in FIG. 3A, wherein the activity of MALT1 protease is measured with cellular activity-based assay, as defined elsewhere herein. In the assay, active MALT1 was detected in parental and TRAF6 KO Jurkat cells after labeling of extracts with biotin-MALT1 ABP pulldown before Western Blot (WB).

Quantification was done by determining the ratio of active (PD) to total (lysate) MALT1 from three independent experiments. Furthermore, T-cell specific deletion of TRAF6 in mice (herein also referred to as Traf6-ΔT) also resulted in constitutive MALT1 protease cleavage of MALT1 substrates, as shown for example in FIG. 4 (For in vivo modification of MALT1 and TRAF6 in mouse models see Example 2). The inventors here show that a consistently augmented cleavage of MALT1 substrates CYLD, HOIL-1, Regnase-1, Roquin-1/2 was evident in MALT1-TBM-T mutant mice (i.e MALT1 mutants unable to bind TRAF6, as defined elsewhere herein) and also in Traf6-ΔT mutant mice (FIG. 4A and FIG. 4B respectively).

As disclosed herein above, TRAF6 may be inactivated, resulting in constitutively active MALT1 protease, by TRAF6 mutations that render the TRAF6 E3 ligase inactive.

In a further preferred embodiment, MALT1 protease activity is rendered constitutive active by: (i) inactivating the C-terminal MATH domain of TRAF6 corresponding to position 350-499 of the TRAF6 protein shown in SEQ ID NO:3, (ii) substituting D→K at a position in a TRAF6 protein corresponding to position 57 of the TRAF6 protein shown in SEQ ID NO:3, (iii) substituting C→A at a position in a TRAF6 protein corresponding to position 70 of the TRAF6 protein shown in SEQ ID NO: 3, (iv) substituting an amino acid at a position in a TRAF6 protein corresponding to position 72 of the TRAF6 protein shown in SEQ ID NO:3, wherein the amino acid substitution is selected from the group consisting of I→D, I→A, I→K, and I→F, (v) substituting an amino acid at a position in a TRAF6 protein corresponding to position 74 of the TRAF6 protein shown in SEQ ID NO: 3, wherein the amino acid substitution is selected from the group consisting of L→H, L→E, L→K, and L→R, or (vi) substituting R→A at a position in a TRAF6 protein corresponding to position 88 of the TRAF6 protein shown in SEQ ID NO: 3 and substituting F→A at a position in a TRAF6 protein corresponding to position 118 of the TRAF6 protein shown in SEQ ID NO: 3. In a preferred embodiment, the MALT1 protease is rendered constitutive active by inactivating a protein essential for the regulation of TRAF6 E3 ligase activity such as UBC13 and/or UEV1A E2 enzymes. As disclosed herein, said amino-acid substitutions may be obtained via mutagenesis. As a result of inactivation of TRAF6 gene (here, by TRAF6 mutations that render the TRAF6 E3 ligase inactive) a constitutive, increased activity of MALT1 is achieved, as demonstrated by an augmented cleavage of MALT1 substrates, as defined elsewhere herein. Effects of TRAF6 E3 ligase inactivation are also shown in FIG. 3B. Here, the inventors show TRAF6 KO Jurkat T-cells, which were reconstituted with mock, TRAF6 WT or TRAF6 E3 ligase inactive mutant (here, TRAF6 C70A mutant). Said cells were analyzed for MALT1 substrate cleavage (WB) with and without P/I stimulation. Active MALT1 was detected by biotin-MALT1 ABP pulldown. Only TRAF6 WT was able to counteract chronic MALT1 protease activity, demonstrating that TRAF6 E3 ligase activity keeps the MALT1 protease in an inactive state. The TRAF6 C70A mutation prevents binding to UBC13, the essential auxiliary factor for TRAF6 E3 ligase activity.

As disclosed herein above, TRAF6 may be inactivated, resulting in constitutively active MALT1 protease, by TRAF6 mutations that abolish TRAF6 oligomerization. As it is known to the skilled artisan, in chemistry and biochemistry, an oligomer is a molecule that consists of a few similar or identical repeating units which are referred to as monomers. In the context of the present invention the word oligomer refers to multiple folded protein subunits or “monomers” (in the present case the TRAF6 protein) in a multi-subunit complex (in this case each subunit/monomer consisting of a TRAF6 protein). The word oligomer encompasses both dimers (formed by two TRAF6 proteins associating with each other) to larger homo-oligomers (more than two TRAF6 proteins associating with each other). In the context of the invention TRAF6 oligomers are formed via the association of two or more TRAF6 proteins via their MATH domain, via its RING-ZF1 domain or via its CC-MATH (=CC (aa287-350)+MATH (aa350-499)) domain.

Therefore, abolishing of TRAF6 oligomerization is achieved in a preferred embodiment by inactivation of the C-terminal MATH domain of TRAF6. As disclosed herein, inactivation may refer to introduction of mutations leading to amino acid substitutions which cause a loss of function of the domain of a protein/or protein in question. Said amino acid substitutions may be obtained via mutagenesis, as defined elsewhere herein. In this context, “inactivating” the C-terminal MATH domain of TRAF6 refers to the introduction of mutations in the TRAF6 gene encoding for TRAF6 protein, resulting in amino-acid substitution in the TRAF6 MATH domain, wherein said mutations inactivate the ability of said domain to bind and thereby interact with TRAF6 MATH domain, thereby impeding the TRAF6-TRAF6 interaction (i.e. oligomerization). The MATH domain is necessary and sufficient for self-association of TRAF6, and it spans the amino acid positions 350-499 of the TRAF6 protein shown in SEQ ID NO: 3. Accordingly, a MATH domain of TRAF6 of the present invention refers to the amino acid positions 350-499 of the TRAF6 protein shown in SEQ ID NO: 3.

Abolishing of TRAF6 oligomerization is preferably also achieved by substituting R→A at a position in a TRAF6 protein corresponding to position 88 of the TRAF6 protein shown in SEQ ID NO: 3 and substituting F→A at a position in a TRAF6 protein corresponding to position 118 of the TRAF6 protein shown in SEQ ID NO: 3. Abolishing of TRAF6 oligomerization is preferably also achieved by substituting F→A at position 118, F→A at position 122 and F→Y at position 118 of the TRAF6 protein shown in SEQ ID NO: 3.

Effects of TRAF6 oligomerization mutant are also shown in FIG. 3C. Here, the inventors show TRAF6 KO mutant Jurkat T-cells, reconstituted with TRAF6 WT, or R88A/F118A oligomerization mutant. Only TRAF6 WT was able to counteract constitutive MALT1 protease activity, demonstrating that TRAF6 oligomerization keeps the MALT1 protease in an inactive state.

According to the invention, the cell of the immune system, defined elsewhere herein, is preferably additionally modified to comprise a suicide gene. Here, by “suicide gene” it is meant a gene which will cause a cell to be prone to kill itself through apoptosis upon treating the patient with a drug that activates the suicide gene if required during therapeutic regimen. In particular, in the context of the present invention, the suicide gene will cause the cell of the invention—namely a cell of the immune system transduced or transfected with a TCR or CAR and further modified to render its MALT1 protease activity constitutive active—to kill itself via apoptosis in a therapeutically controllable manner. A suicide gene may be introduced in the cell of the invention by means of recombinant DNA technology. For this purpose, the cell may be transformed with a cloning vector that includes a nucleic acid molecule encoding a suicide gene as defined herein. Preferably, said suicide gene upon its induction with an appropriate activator drug will induce the cell of the invention to undergo apoptosis after successful therapeutic activity of the cell of the invention, but before an overshooting response of the immune system can be triggered by the cell of the invention. Therefore, the suicide gene strategy is intended to prevent therapy-caused autoimmune and auto-inflammatory effects. In particular, the suicide gene can be, for example, a Herpes simplex virus thymidine kinase (HSV-tk) that is activated to kill a cell by administering specific nucleoside analogues such as ganciclovir (GCV) which is modified by HSV-tk for the inhibition of DNA synthesis and cell death induction. Another example for a suicide gene is a modified human caspase-9 (iCasp9) which can be induced to trigger cell death upon administration of a chemical compound (AP1903) that causes dimerization of caspase-9 and its activation as an initiator caspase for the induction of apoptosis.

The present invention further refers to the cell of the immune system as described elsewhere herein or a composition comprising such cell for use as a medicament. Hence, the cell of the immune system as described elsewhere herein or a composition comprising such cell of the immune system can also be used for therapy, i.e. the treatment of a disease. In a preferred embodiment of the invention, the cell of the immune system according to the invention is for use in adoptive T cell therapy. Preferably, the cell of the immune system according to the invention is for use in a method of treating cancer. Accordingly, the present invention relates to a cell of the immune system as described elsewhere herein for use in a method of preventing and/or treating cancer in a subject. In preferred embodiments, the present invention relates to a cell of the immune system or a composition comprising such cell of the immune system for use as a medicament, wherein said cell is a cell of the immune system transduced or transfected with a T cell receptor (TCR) or chimeric antigen receptor (CAR), further modified to render its MALT1 protease activity constitutive active. In particular, according to the use of the cell of the present invention as a medicament, said cell is (after being genetically modified as defined elsewhere herein to express synthetic receptors with specificities against tumor antigens, and being expanded and tested in vitro) transferred back to the patient, wherein said cell will bind to specific tumor-associated antigens, causing an anti-tumor immune response, leading to tumor cell lysis. Additionally, said cell will not enter an exhaustion state, as defined elsewhere herein, as a consequence of the constitutive active MALT1 protease activity, defined elsewhere herein, and as can be seen for example in FIG. 5 and FIG. 6.

As used herein, the term “treat”, “treating” or “treatment” means to reduce (slow down (lessen)), stabilize or inhibit or at least partially alleviate or abrogate the progression of the symptoms associated with the respective disease. Thus, it includes the administration of said cell of the immune system, preferably in the form of a medicament, to a subject, defined elsewhere herein. Those in need of treatment include those already suffering from the disease, here cancer. Preferably, a treatment reduces (slows down (lessens)), stabilizes, or inhibits or at least partially alleviates or abrogates progression of a symptom that is associated with the presence and/or progression of a disease or pathological condition. “Treat”, “treating”, or “treatment” refers thus to a therapeutic treatment. In particular, in the context of the present invention, treating or treatment refers to an improvement of the symptom that is associated with cancer, as defined elsewhere herein. The term “subject” when used herein includes mammalian subjects. Preferably the subject of the present invention is a mammal, including human. In some embodiment the mammal is a mouse. A subject also includes human and veterinary patients. Where the subject is a living human who may receive treatment for a disease or condition as described herein, it is also addressed as a “patient”. Those in need of treatment include those already suffering from the disease.

In particular, the cell of the present invention may be used in adoptive cell therapy, preferably in adoptive T cell therapy. Adoptive cell therapy as used herein and also known as cellular immunotherapy refers to a form of treatment that uses the cells of the human immune system to eliminate cancer. Some of these approaches involve directly isolating patient's own immune cells and simply expanding their numbers, whereas others involve genetically modifying the immune cells to enhance their cancer-fighting capabilities. The adoptive cell therapy as used herein makes use of the cell of the invention. In particular, in the context of the invention the adoptive cell therapy entails collecting and isolating a T cell, NK cell, NKT cell or a macrophage from a subject, subject said cells to viral transfer of a TCR or CAR, in addition to gene editing to render MALT1 protease constitutively active, and subsequently expand and transferring said cells back to the subject, as defined elsewhere herein. MALT1 protease, which is constitutively active, has the effect of enhancing the activity of a cell transduced or transfected with a TCR or CAR. As a consequence, these MALT1-TCR/CAR-edited T cells induce a strong, specific and long-lasting immune response by permanently activated MALT1. The effector T cells thus remain more reactive, MALT1 does not have to be activated first by antigen receptor stimulation (TCR/CAR-tumor antigen), the effector T cells are not suppressed by regulatory T cells and the T cells do not enter the “exhausted” state.

The present invention also relates to a method for generating the cell according to the invention, comprising modifying said cell to render MALT1 protease constitutively active. Modifying MALT1 protease activity constitutively active is defined elsewhere herein. According to the present invention, the method of generating a cell of the invention, comprising modifying the immune cell to render MALT1 protease constitutively active is preferably preceded by the step of transducing or transfecting a T cell, NK cell, NKT cell or a macrophage with a heterologous TCR or CAR. Accordingly, the present invention further provides a method of manufacturing a CAR or TCR-expressing cell, comprising introducing nucleic acid encoding a CAR or TCR into a cell such that said nucleic acid (or CAR/TCR-encoding portion thereof) integrates into the genome of the cell. For example, the cell may be subjected to viral transfer of TCR or CAR. Additionally, the cell may be subjected to gene editing to generate the constitutively active MALT1, defined elsewhere herein. The skilled artisan is aware of methods for transducing or transfecting cells. In the context of the invention, primary human T cells, NK cells, NKT cells or macrophages can be modified using viral and non-viral vectors to promote the specific targeting of cancer cells via the introduction of heterologous T-cell receptors (TCRs) or chimeric antigen receptors (CARs). When non-viral vectors are used, the cells are said to be transfected. The term “transfection” refers to the process of introducing naked or purified nucleic acids into eukaryotic cells. Said naked or purified nucleic acids can be in the form of a vector. Accordingly, in the context of the invention, oligonucleotides encoding the heterologous T-cell receptors (TCRs) or chimeric antigen receptors (CARs) are transfected into said human T cells, NK cells, NKT cells or macrophages. Alternatively, a cell of the human immune system can be modified by transduction. The term “transduction” refers to the process by which foreign DNA is introduced into a cell by a virus or viral vector. In particular, in the context of the present invention viral transduction of TCR or CAR into the cell of the human immune system is preferred. Viral vectors suitable for said transfection are known to the skilled artisan. In the context of the present invention, the method of generating the cells of the invention comprises the steps of a) collection and isolation of cells of the human immune system from a subject, b) transduction or transfection of said cells with TCR or CAR, c) gene editing said transfected cells, to render the MALT1 protease of said cells constitutively active, wherein the constitutive activity of said MALT1 protease is determined as defined elsewhere herein. Exemplary transduction methods are summarized in Example 11. Preferably, the cell of the human immune system as used herein is a primary human T cell, NK cell, NKT cell or macrophage.

The present invention also relates to an in vitro method of enhancing the activity of a cell of the human immune system transduced or transfected with a T cell receptor (TCR) or chimeric antigen receptor (CAR), comprising modifying said cell in that MALT1 is rendered constitutive active. In a preferred embodiment of the invention, said in vitro method foresees that said cell of the immune system is a T cell, NK cell, NKT cell, or a macrophage. Preferably, said in vitro method foresees that the activity of a cell of the human immune system is monitored by increased activation markers, such as T cell activation markers and/or increased cytokine production. Accordingly, said in vitro method comprises the steps of a) transduction or transfection of a cell of the human immune system with TCR or CAR, b) gene editing said transfected cells, to render the MALT1 protease of said cells constitutively active, wherein the constitutive activity of said MALT1 protease is determined as defined elsewhere herein, c) expand the tumor reactive cell of the human immune system, and d) testing the activity of the resulting engineered cell of the human immune system. Gene editing techniques are known to the skilled artisan, in particular, regarding the in vitro method of the invention, gene editing of the transfected cells to render the MALT1 protease of said cells constitutively active (step b) may preferably be achieved by CRISPR/Cas9-based gene editing techniques, including for example Homology Directed Repair (HDR) template-mediated gene modification or Cytosine Base Editing (CBE) or Adenine Base Editing (ABE) techniques. Gene editing to render the MALT1 protease constitutively active may also be achieved by other known techniques such as double strand break repair, use of engineered nucleases such as Zinc finger nucleases (ZFNs), transcription-activator like effector nucleases (TALEN), or meganucleases. Preferably, said cell of the immune system is a T cell, NK cell, NKT cell, or a macrophage, as defined elsewhere herein. In the context of the in vitro method of the present invention, the activity of a cell of the human immune system as defined herein is monitored by increased activation markers, such as T cell activation markers and/or increased cytokine production. For example, said increased T cell activation markers may be markers selected from the group consisting of CD69, CD44^{high}, CD62^{low} CD4 and CD8 T_{EM} cells (see FIG. 4C). Upregulation of T cell activation markers in T cells lacking TRAF6 (Traf6-ΔT mice) relies on MALT1 protease activity, as revealed by downregulation of these markers with the use of the potent and selective MALT1 protease inhibitor MLT-985 (FIG. 5). Further, upregulation of T cell activation markers in T cells lacking MALT1-TRAF6 interaction (Malt1 TBM mice) is relying on MALT1 protease activity, as revealed by downregulation of these markers in T cells after genetic mutation of MALT1 protease activity in mice with mutations in the T6BM as well as paracaspase (PM: paracaspase mutant) (Malt1 TBM/PM mice) (FIG. 6). Further, a T-cell activation marker may also be an increased expression of T cell activator genes controlled by MALT1 protease in CD4+ and CD8+ T cells, such as I_{K}BNS (FIG. 4D) or ICOS (FIG. 4E). Expression of both proteins is controlled on the post-transcriptional level by the MALT1 substrates Regnase-1 and Roquin-1/2 and thus TCR/CD28-induced upregulation of I_{K}BNS and ICOS was suppressed by the selective MALT1 protease inhibitor MLT-985 (FIG. 5D).

Accordingly, MALT1 protease inhibitors or genetic inactivation are able to revert the MALT1 constitutive activity. Further, E→D in MALT1-TRAF6 binding motif 2 (T6BM2) corresponding to position 806 of the MALT1A protein shown in SEQ ID NO: 1 or at a position in a MALT1B protein corresponding to position to position 795 of the MALT1B protein shown in SEQ ID NO: 2 leads to constitutive MALT1 protease activity (FIG. 2C). The same mutation inserted into the genomic locus of the Malt1 gene in mice (Malt1 TBM2 mice) also leads to upregulation of T cell activation markers (FIGS. 7A and B). Furthermore, or alternatively, the activity of a cell of the human immune system according to the invention may also be monitored by monitoring cytokine production. In particular, a cell according to the invention may lead to an increased cytokine production. In this context, increased cytokine production may refer to increased serum concentrations of IL-2, IL-4, IL-6, IL-10, IL-17, GM-CSF, INFγ, and TNFα. Preferably, upregulation of these cytokines may be shown, which relies on MALT1 protease activity in Malt1 TBM mice and thus is lost in Malt1 TBM/PM double mutant mice (FIG. 6).

In particular, with the term “increased”, when referring to cytokine production it is meant an increase of serum concentration of cytokines of about 200-5000% as measured by flow cytometry bead array, when compared to cytokine production in a cell of the immune system which is transduced or transfected with a T cell receptor (TCR) or chimeric antigen receptor (CAR), but which is not further modified to render its MALT1 protease activity constitutive active. The cytokine production may be measured, for example, via flow cytometry using a cytometric bead array kit, as described in Example 7.

According to the present invention, “enhancing the activity” of a cell of the immune system transduced or transfected with a TCR or a CAR, refers to the prevention of the exhaustion state of the immune system, namely cells of the immune system which, after being confronted with an antigen over a longer period of time, show a decreased activity or state of exhaustion wherein characteristic immune checkpoint molecules (PD-1, TIGIT, LAG-3, etc.) are expressed and anti-tumor effector cytokines (IFNγ, TNFα, etc.) are downregulated. In the context of the invention said prevention of the exhaustion state (i.e “enhancing the activity”) of a cell of the immune system transduced or transfected with a TCR or a CAR is achieved by additionally modifying said cell in that MALT1 is rendered constitutive active, as defined elsewhere herein.

Accordingly, the present invention also relates to an in vitro use of constitutive active MALT1 for enhancing the activity of a cell transduced or transfected with a T-cell receptor (TCR) or chimeric antigen receptor (CAR). As used herein, enhancing the activity of said cell refers to the fact that constitutive active MALT1 in TCR-T-cells or CAR-T-cells, has the consequence that specifically these MALT1-TCR/CAR-edited T cells display an augmented cleavage of MALT1 substrates, as defined elsewhere herein, and an increase in activation markers, such as T cell activation markers and/or increased cytokine production, as defined elsewhere herein, by permanently activated MALT1 in the absence of an external stimulus (i.e. TCR ligation by a cognate antigen as defined elsewhere herein). As a consequence, the MALT1-TCR/CAR-edited T cells thus remain more reactive, MALT1 does not have to be activated first by antigen receptor stimulation (TCR/CAR-tumor antigen), the T effector cells are not suppressed by regulatory T cells and the T cells do not fall into a “state of exhaustion”, defined elsewhere herein.

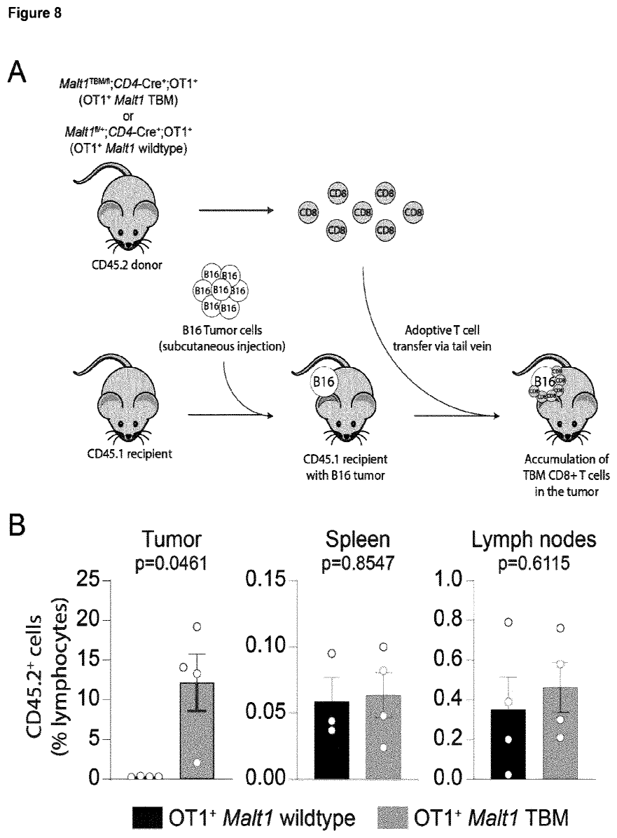

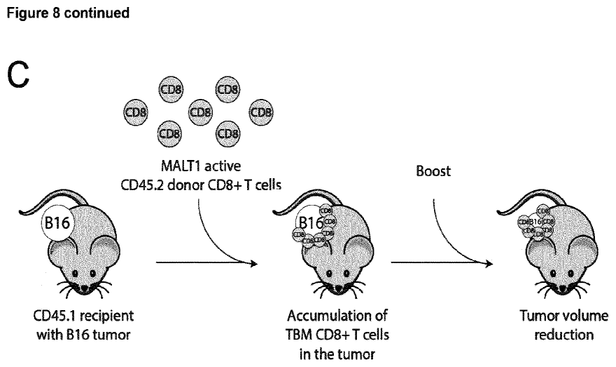

The present invention also relates to the cell of the immune system as described elsewhere herein or a composition comprising such cell for use in a method of enriching the cell at a target site in a subject. Accordingly, it is foreseen that the immune cell according to the invention can be used in a method which allows that the cell is enriched at a target site. Preferably, it is foreseen that the method comprises that the immune cell according to the invention is provided to a subject, and thereby an enrichment of the cell is achieved at the target site. Preferably, a target site can be understood as a distinct site, a tissue or a structure, which is preferred to be approached by the immune cells of the invention. Preferably, it is foreseen that such a target site may be any desired site such as a tumor, e.g. a malignant tumor or a benign tumor, a tissue, an infection site, or an infected tissue, or a tissue pattern. The inventors could show (Example 20 and FIG. 8) that the immune cells of the invention accumulate at the site of a tumor, whereas control cells having wildtype MALT1 do not accumulate. This accumulation or enrichment of the cells of the invention provide the presence of respectively activated immune cells—due to the constitutive active MALT1 protease activity—in closest vicinity to the tumor. The enhanced presence and enrichment of said activated cells of the invention is capable of exerting direct influence on the target site, which means that any effector function of the cell of the invention is applied to the target site. Such an effector function will result in e.g. an anti-tumor response. Therefore, the immune cell of the present invention is able to provoke a beneficial effect in the treatment of cancer or any other disease, which requires the presence of activated immune cells. This provides, in particular, advantages for the use of the cell of the invention in adoptive T cell therapy. As shown in Example 20, CD8+ T cells are enriched specifically at the tumor site, which is a melanoma. The concentrated presence and enrichment of said CD8+ T cells allows the provision of distinct and elevated anti-tumor responses.

In a further embodiment, the present invention also relates to the cell of the immune system as described elsewhere herein or a composition comprising such cell for use in a method of enriching the cell at a target site in a subject, wherein the method further comprises providing an agent for immune system stimulation. In accordance with the present invention, an agent for immune system stimulation may be selected from a biologic or chemical agent, such as a protein, preferably an antigenic peptide; a cytokine; an antibody; a nucleic acid, preferably an mRNA vaccine, or oligonucleotides; an inorganic or organic immune adjuvant, preferably aluminium, Freund's adjuvant, bacterial products, paraffin oils; and any other agent that modulates the immune response and enhances the effector function of the cell of the invention. The effector function of the cell of the invention can be understood in one embodiment as the cytotoxic killing of tumor cells. Distinct examples of an agent for immune system stimulation are AS01 (Adjuvant System), AS02, AS03, AS04, CpG-oligonucleotide, IC31, ISCOMATRIX, MF59, MPL, QS-21, and virosomes.

The present invention is further characterized by the following items:

1. Cell of the human immune system transduced or transfected with a T cell receptor (TCR) or chimeric antigen receptor (CAR), said cell being further modified to render its MALT1 protease activity constitutive active.

2. The cell of item 1, wherein said cell of the human immune system is a T cell, NK cell, NKT cell, a B cell or a macrophage.

3. The cell of item 2, wherein said T cell is a CD8+ T cell, a CD4+ T cell or a Treg cell.

4. The cell of any one of the preceding items, wherein the constitutive active MALT1 protease activity of said cell is characterized by increased MALT1 protease activity in comparison to MALT1 protease activity of a cell of the immune system transduced or transfected with a T cell receptor (TCR) or chimeric antigen receptor (CAR), but which is not further modified to render its MALT1 protease activity constitutive active.

5. The cell of any one of the preceding items, wherein MALT1 protease activity is determined by cleavage of MALT1 substrates such as Regnase-1, Roquin1, Roquin2, N4BP1, HOIL-1/RBCK1, CYLD, A20/TNFAIP3, BCL10, MALT1 or RelB.

6. The cell of any one of the preceding items, wherein MALT1 protease activity is determined by cellular activity-based assay utilizing a MALT1 activity-based probe (ABP) such as Biotin-LVSR-AOMK, Biotin-LRSR-AOMK or BODIPY-LVPipR-AOMK

7. The cell of any one of the preceding items, wherein MALT1 protease activity is rendered constitutive active by abolishing MALT1 interaction with TRAF6 or by deletion of TRAF6.

8. The cell of item 7, wherein MALT1 interaction with TRAF6 is abolished by inactivating the TRAF6 binding motif 1 (T6BM1) of human MALT1A and/or the TRAF6 binding motif 2 (T6BM2) of human MALT1A or human MALT1B.

9. The cell of item 8, wherein T6BM1 encompasses amino acids 314 to 319 of human MALT1A shown in SEQ ID NO: 1 and T6BM2 encompasses amino acids 804 to 809 of human MALT1A shown in SEQ ID NO: 1 or amino acids 793 to 798 of human MALT1B shown in SEQ ID NO: 2.

10. The cell of any one of items 7 to 9, wherein MALT1 protease activity is rendered constitutive active by substituting E for another amino acid, preferably E→A or E→D at a position in a MALT1A protein corresponding to position 316 in SEQ ID NO: 1

11. The cell of any one of items 7 to 9, wherein MALT1 protease activity is rendered constitutive active by substituting E for another amino acid, preferably E→A or E→D at a position in a MALT1A protein corresponding to position 806 of the MALT1A protein shown in SEQ ID NO: 1 or at a position in a MALT1B protein corresponding to position to position 795 of the MALT1B protein shown in SEQ ID NO: 2.

12. The cell of any one of the preceding items, wherein MALT1 protease activity is rendered constitutive active by (i) substituting Y→A at a position in a MALT1A protein corresponding to position 657 of the MALT1A protein shown in SEQ ID NO: 1;

(ii) substituting an amino acid at a position in a MALT1A protein corresponding to position 506 of the MALT1A protein shown in SEQ ID NO: 1, wherein the amino acid substitution is selected from the group consisting of L→A, L→G, and L→K, (iii) substituting N→A at a position in a MALT1A protein corresponding to position N508 of the MALT1A protein shown in SEQ ID NO: 1, or (iv) substituting Y→A at a position in a MALT1A protein corresponding to position Y367 of the MALT1A protein shown in SEQ ID NO: 1.

13. The cell of any one of the preceding items, wherein MALT1 protease activity is rendered constitutive active by (i) substituting Y→A at a position in a MALT1B protein corresponding to position 646 of the MALT1B protein shown in SEQ ID NO: 2, (ii) substituting Y→A at a position in a MALT1B protein corresponding to position L495 of the MALT1B protein shown in SEQ ID NO:2, (iii) substituting Y→A at a position in a MALT1B protein corresponding to position N495 of the MALT1B protein shown in SEQ ID NO:2, or (iv) substituting Y→A at a position in a MALT1B protein corresponding to position Y356 of the MALT1B protein shown in SEQ ID NO:2.

14. The cell of any one of the preceding items, wherein MALT1 protease activity is rendered constitutive active by inactivating TRAF6 through inactivating TRAF6 gene expression, rendering TRAF6 E3 ligase inactive or abolishing TRAF6 oligomerization.

15. The cell of item 14, wherein MALT1 protease activity is rendered constitutive active by (i) inactivating the C-terminal MATH domain corresponding to position 350-499 of the TRAF6 protein shown in SEQ ID NO: 3, (ii) substituting D→K at a position in a TRAF6 protein corresponding to position 57 of the TRAF6 protein shown in SEQ ID NO:3, (iii) substituting C→A at a position in a TRAF6 protein corresponding to position 70 of the TRAF6 protein shown in SEQ ID NO: 3, (iv) substituting an amino acid at a position in a TRAF6 protein corresponding to position 72 of the TRAF6 protein shown in SEQ ID NO:3, wherein the amino acid substitution is selected from the group consisting of I→D, I→A, I→K, and I→F, (v) substituting an amino acid at a position in a TRAF6 protein corresponding to position 74 of the TRAF6 protein shown in SEQ ID NO: 3, wherein the amino acid substitution is selected from the group consisting of L→H, L→E, L→K, and L→R, or (vi) substituting R→A at a position in a TRAF6 protein corresponding to position 88 of the TRAF6 protein shown in SEQ ID NO: 3 and substituting F→A at a position in a TRAF6 protein corresponding to position 118 of the TRAF6 protein shown in SEQ ID NO: 3.

16. The cell of any one of the preceding items, wherein MALT1 protease activity is rendered constitutive active by inactivating a protein essential for the regulation of TRAF6 E3 ligase activity, such as the E2 enzymes UBC13 and UEV1A.

17. The cell of any one of the preceding items, wherein MALT1 substrates are genetically inactivated.

18. The cell of any one of the preceding items, wherein said cell is additionally modified to comprise a suicide gene.

19 Cell of any one of items 1 to 18 for use as a medicament.

20. Cell of any one of items 1 to 18 for use in adoptive T cell therapy.

21. Cell of any one of items 1 to 18 for use in a method 18 treating cancer.

22. A method for generating a cell of any one of items 1 to 18, comprising modifying a cell to render MALT1 protease activity constitutive active.

23. The method of item 22, wherein said modifying step is preceded by the step transducing or transfecting a T cell, NK cell, NKT cell or a macrophage with a heterologous TCR or CAR.

24. An in vitro method of enhancing the activity of a cell of the human immune system transduced or transfected with a T cell receptor (TCR) or chimeric antigen receptor (CAR), comprising modifying said cell in that MALT1 is rendered constitutive active.

25. The method of item 24, wherein said cell of the immune system is a T cell, NK cell, NKT cell, or a macrophage.

26. The method of item 24 or 25, wherein activity of a cell of the human immune system is monitored by increased activation markers, such as T cell activation markers and/or increased cytokine production.

27. In vitro use of constitutive active MALT1 for enhancing the activity of a cell transduced or transfected with a T cell receptor (TCR) or chimeric antigen receptor (CAR).

### EXAMPLES OF THE INVENTION

The following examples illustrate the invention. These examples should not be construed as to limit the scope of this invention. The examples are included for purposes of illustration and the present invention is limited only by the claims.

Example 1—Detection of active MALT1 by activity-based probes (ABP). Generation and application of biotin labeled MALT1 activity-based probes (MALT1-ABPs) has been described previously (Eitelhuber, A. C. et al. Activity-based probes for detection of active MALT1 paracaspase in immune cells and lymphomas. Chem Biol 22, 128-138, doi10.1016/j.chembio.2014.10.021 (2015)). Jurkat T-cells (3×10⁷) were washed with PBS and lysed in 600 μl co-IP buffer without protease inhibitors for 25 min at 4° C. Cleared lysates (>20.000×g, 4° C., 10 min) were used to collect lysate control (60 μl) or incubated with High Capacity Streptavidin Beads (Thermo Fisher, 12 μl) for 1 hour at 4° C. for pre-clearing (490 μl). Beads were pelleted (1700×g, 2 min, 4° C.) and 420 μl of supernatant mixed with biotin-labeled MALT1-ABP at a final concentration of 0.1 μM. After 50 min rotating at RT, High Capacity Streptavidin Beads (Thermo Fisher, 15 μl) were added and samples incubated for 1-2 hours at 4° C. (rotating). Beads were collected (1700×g, 2 min, 4° C.), washed 3× with co-IP buffer without protease inhibitors, resuspended in 22 μl 2xSDS loading dye, boiled at 95° C. for 7 min and analyzed by WB.

Example 2—in vivo modification of MALT1 and TRAF6 in mouse models. Malt1 TBM (TBM: TRAF6 binding mutant) mice carrying genomic missense mutations in TRAF6 binding motif 1 and 2 (T6BM1 and 2) were generated by CRISPR/Cas9 mutagenesis in R1/E ES cells and subsequent embryo injection (see below). Malt1 TBM mice express the mutant variants MALT1A E325A; E814A and MALT1B E803A. Malt1 TBM2 mice were generated by CRISPR/Cas9 genomic mutation of T6BM2 in zygotes of mice. Malt1 TBM2 mice express the mutant variants MALT1A E814D and MALT1B E803D. Malt1 floxed (fl) mice were derived from the EUCOMM ES cell clone Malt1^{tm1a(EUCOMM)Hmgu} (HEPD0618_3_D10), which was injected into blastocysts and transferred into foster mothers and the IRES-lacZ/Neo cassette was deleted by crossing to ROSA26-FLPe delete mice (Tg(ACTFLPe)9205Dym) (Rodriguez, C. I. et al., High-efficiency deleter mice show that FLPe is an alternative to Cre-loxP. Nat Genet 25(2): 139-40 (2000)). For conditional expression of the Malt1 TBM allele in T-cells, Malt1^{TBM/+} were crossed to CD4-Cre (CD4-Cre (Tg(CD4-cre) 1Cwi) (Lee, P. P. et al., A critical role for dnmt1 and DNA methylation in T cell development, function, and survival. Immunity 15(5):763-74 (2001)) to yield Malt1^{TBM/+}; CD4-Cre, which were paired to Malt1^{ﬂ/ﬂ}mice to yield Malt1^{TBM/ﬂ}; CD4-Cre. Malt1^{TBMPM/+} mice were generated by CRISPR/Cas9 genomic mutation of the paracaspase active site (C472A) in Malt1^{TBM/+} mice to yield simultaneous TBM1/2 and PM (paracaspase mutation) mutations in MALT1. For Traf6^{ﬂ/ﬂ}; CD4-Cre (Traf6-ΔT) mice, frozen embryos of Traf6^{tm2a(EUCOMM)Wtsi} (EMMA ID EM:08446; Infrafrontiers Biocenter Oulu) were transferred into foster mothers and the IRES-lacZ/Neo cassette was deleted by crossing to ROSA26-FLPe-deleter mice. Traf6^{ﬂ/ﬂ}mice were crossed to CD4-Cre transgenic mice for T-cell specific deletion of exons 4 and 5 of the Traf6 locus.

Example 3—Design of sgRNA and HDR templates for generation of Malt1^{TBM/TBM} mice. For mutagenesis of the two TRAF6 binding motifs (T6BMs) in MALT1 (T6BM1 in exon 7 and T6BM2 in exon 17) targeting sequences for single guide (sg) RNAs were designed using the GPP sgRNA Designer provided by the BROAD Institute (Doench, J. G. et al., Optimized sgRNA design to maximize activity and minimize off-target effects of CRISPR-Cas9. Nature biotechnology, 34(2), 184-191. doi: 10.1038/nbt.3437 (2016)) (https://portals.broadinstitute.org/gpp/public/analysis-tools/sgrna-design). The respective coding DNA sequences were cloned into pX458 plasmid (Addgene #48138). Short single stranded oligodeoxyribonucleotides (ssODNs) homology directed repair (HDR) templates were synthesized that introduce TRAF6 binding mutation 1 and 2 (TBM1 and TBM2) and alterations in the PAM sequences by silent mutations to prevent multiple Cas9 cleavage events at the locus in the Malt1 gene. Additional silent mutations in the region of the TBM mutations were inserted to enable differential PCR amplification by WT- and mutation-specific PCR primers for the identification of targeted alleles in ES cell clones and genotyping.

Example 4—Generation of Malt1^{TBM/TBM} mice. ES cell clone 3-3/E10 containing correct homozygous alterations in both T6BMs was injected into 8-cell C57BI6/Ncrl wildtype embryos by utilizing laser-assisted injection technology (Poueymirou, W. T. et al., F0 generation mice fully derived from gene-targeted embryonic stem cells allowing immediate phenotypic analyses, 25(1), 91-99, doi: 10.1038/nbt1263 (2007)). Donor embryos were generated by natural mating. Two to three hours after ES-cell injection, 10-12 embryos were transferred into the oviduct of a pseudogravide Crl:CD1(IDR) female, and chimeric males were born with brown fur (>90% chimerism). After crossing to C57BL/6 animals, germline transmission and brown F1 generation yielded heterozygous Malt1^{TBM/+} and homozygous Malt1^{TBM/TBM} mice upon crossing. Genotypes were identified by differential PCR and mutations were verified by genomic sequencing. The complete Malt1 CDS was sequenced to verify that no additional mutations changing the coding sequence were found.

Example 5—Immune cell phenotyping by flow cytometry. Lymphocyte populations were analyzed from single cell suspensions prepared from murine tissues (spleen, lymph nodes, thymus, or bone marrow). Tissue was collected from mice and meshed, treated with red blood cell lysis buffer (Miltenyi, 130-094-183), and 1 million cells were plated per staining. Cells were washed twice with PBS (350×g, 5 min, 4° C.) and dead cells were stained using eFluor780 dye (eBioscience, 65-0865-18, 1:1000 in PBS, 30 min, 4° C.). Cells were washed with FCM buffer (3% FCS in PBS) and treated with anti-CD16/CD32 (Fc-block, eBioscience, 14-0161-85, 1:200 in FCM buffer, 20 min, RT). Supernatant was discarded and cells were stained with fluorescent antibodies for 20 min at RT. Staining was performed with anti-CD3-PECy7 (1:300, 25-0031-82), anti-CD8a-FITC (1:100, 11-0081-85), anti-CD4-PE (1:300, 12-0042-85), anti-CD4-PerCP-Cy5.5 (1:300, 45-0042-82), anti-CD44-PECy7 (1:400, 25-0441-82), anti-CD44-FITC (1:300, 11-0441-81), anti-CD62L-APC (1:300, BD Pharmingen, 553152), anti-0x40-PECy7 (1:200, 25-1341-80), anti-ICOS-FITC (1:200, 11-9949-82) and anti-I_{K}BNS (1:10, 41, HMGU). All antibodies are from eBiosciences except where indicated. For intracellular staining of FoxP3, cells were permeabilized and fixed (eBiosciences, 00-5223-56), washed once with permeabilization buffer (eBiosciences, 546 00-8333-56) and stained for 30 min with anti-FoxP3-PE (1:100, 12-5773-82) in permeabilization buffer. Cells were washed with sorting buffer and analyzed using an Attune Acoustic Focusing Cytometer (Thermo Fisher) or a Fortessa Cytometer (Becton Dickinson, Franklin Lakes, NJ).

Example 6—Analyses of I_{K}BNS and ICOS expression by flow cytometry. For intracellular I_{K}BNS and extracellular ICOS staining, primary murine splenocytes (1×10⁶) were collected, centrifuged (300×g, 5 min, 4° C.) and washed twice with PBS. Afterwards, live/dead staining (eBioscience Fixable Viability Dye eFluor 780, 1:1000 in PBS) was added for 30 min at 4° C. Cells were washed again with PBS, fixed in 2% PFA for 20 min at 4° C. and permeabilized in Saponin buffer (0.5% saponin and 1% BSA in PBS) for 25 min at RT. Unspecific antibody binding was blocked with anti-CD16/32 (1:100 in Saponin buffer, 10 min, 4° C.) before samples were incubated with anti-I_{K}BNS antibody (clone 4C1, rat, 1:10 in Saponin buffer) for 30 min at 4° C. Cells were washed (300×g, 5 min, 4° C.) and stained with a secondary mouse anti rat-AF647 (1:200 in Saponin buffer, 30 min at 4° C.). In parallel, anti-ICOS-FITC antibody in Saponin buffer was added. Samples were again washed (300×g, 5 min, 4° C.) and wells were filled up with Saponin buffer to wash out unbound antibodies (>15 min, RT). For surface staining, anti-CD4-PerCP and anti-CD8-PE was added for 30 min at RT. Samples were again washed with FCM buffer (300×g, 5 min, 4° C.), re-suspended in FCM buffer and acquired on an Attune Cytometer.

Example 7—Analyses of cytokines and autoantibodies. Cytokines in sera of mice were measured according to manufacturer protocol via flow cytometry using a cytometric bead array kit (BD, 562246) and specific beads for the cytokines IL-4 (BD, 562272), IL-6 (BD, 562236), IL-10 (BD, 562263), IL-17 (BD, 562261), INFγ (BD, 562233), and TNFα (BD, 562336).

Example 8—Therapeutic treatment of Traf6-ΔT mice with a MALT1 inhibitor. MLT-985 stock solution was prepared at 40 mg/mL in DMSO and vortexed to solubilize. For injection, stock was diluted 1:20 in DPBS, yielding a crystalline suspension. 8 week old Traf6-ΔT mice were injected i.p. BID at 16 mg/kg in a volume of 200 μL per 25 g body weight over a 10 day period. Suspension was kept at 37° C. prior to each injection. On day 11, lymphocyte populations were analyzed via flow cytometry and Western blot.

Example 9—Cultivation, stimulation and inhibitor treatment of cell lines and primary cells. All cell lines were maintained in humidified atmosphere (37° C., 5% CO₂). Jurkat T-cells were cultured in RPMI 1640 Medium, HEK293T-cells in DMEM. Media were supplemented with 10% fetal calf serum, 100 U/ml penicillin and 100 μg/ml streptomycin (all Gibco). Jurkat T-cells were verified by the Authentication Service of the Leibniz Institute (DSMZ). Primary murine splenocytes were isolated from spleen, treated with Red Blood Cell Lysis Solution (Miltenyi) and CD4 T-cells purified by using the CD4 T-cell isolation kit Il (Miltenyi) by negative magnetic-activated cell sorting (MACS). CD4 T-cells were cultured in primary T-cell medium (RPMI 1640, 100 U/ml penicillin, 100 μg/ml streptomycin, 10% heat inactivated fetal calf serum, 10 mM HEPES PH 7.5, 2 mM L-Glutamine, 1 mM Sodium-Pyruvate, MEM-NEAA (1x), 50 nM β-Mercaptoethanol (all Gibco). Jurkat T-cells or primary murine CD4 T cells were stimulated with Phorbol 12-Myristate 13-Acetate (PMA, 200 ng/ml; Merck)/lonomycin (lono, 300 ng/ml; Calbiochem).

Example 10—Generation of MALT1 KO and TRAF6 KO Jurkat T-cells. Generation of MALT1-deficient Jurkat T-cells has been described earlier (Meininger, I. et al. Alternative splicing of MALT1 controls signalling and activation of CD4(+) T cells. Nat Commun 7, 11292, doi: 10.1038/ncomms11292 (2016)). For generation of TRAF6 KO Jurkat T-cells, sequences coding for sgRNAs targeting in Exon 1 and Exon 2 were cloned into vector px458, and transfected by electroporation in parental Jurkat T-cells using 220 V and 1,000 μF utilizing a Gene Pulser X (BioRad), and sorted for GFP-expressing cells after 2 days using a MoFlow sorting system. After serial dilution and expansion of single clones, KO cell lines were confirmed by protein expression on WB.

Example 11—Lentiviral transduction of Jurkat T-cells. For stable reconstitution of TRAF6- and MALT1-deficient Jurkat T cells, TRAF6 and MALT1 constructs were linked to hΔCD2 by a co-translational processing site T2A (Hadian, K. et al. NF-kappaB essential modulator (NEMO) interaction with linear and lys-63 ubiquitin chains contributes to NF-kappaB activation. J Biol Chem 286, 26107-26117, doi: 10.1074/jbc.M111.233163 (2011).) and introduced into a pHAGE transfer vector. 2×10⁶ HEK293T cells were seeded in 10 cm² dishes and transfected with 1.5 μg psPAX2 (Addgene #12260; gift D. Trono), 1 μg pMD2.G (Addgene #12259; gift D. Trono) and 2 μg transfer vector using X-tremeGENE HP DNA Transfection Reagent (Roche). For transduction, virus-containing supernatant was applied to 5×10⁵ Jurkat T-cells, mixed with Polybrene (8 μg/ml) and incubated for 24 hours. After transduction, cells were washed with PBS, resuspended in RPMI, cultured for ten days and expression of hΔCD2 determined by flow cytometry. Protein expression was confirmed by WB.

Example 12—Preparation of cell lysates. For cellular analysis via WB, Jurkat or primary murine CD4 T-cells (2-3×10⁶) were washed 1× in PBS and lysed in co-immunoprecipitation (co-IP) buffer (25 mM HEPES pH 7.5, 150 mM NaCl, 0.2% NP-40, 10% glycerol, 1 mM DTT, 10 mM NaF, 8 mM β-glycerophosphate, 300 μM sodium vanadate and protease inhibitor cocktail mix (Roche)) for 20 min at 4° C. Lysate controls were mixed with 4× SDS loading dye, boiled for five min at 95° C., separated by SDS-PAGE and analyzed by WB.

Example 13—Western blotting (WB). An electrophoretic semi-dry blotting system was used to transfer SDS-PAGE separated proteins onto PVDF-membranes (Merck Millipore). After transfer, membranes were blocked with 5% BSA (Sigma-Aldrich) or 5% milk (Roth) in PBS-Tween (0.01% Tween) for 1 hour at RT. Primary antibodies were diluted as indicated in 2.5% BSA or milk in PBS-T and membranes incubated overnight at 4° C. Membranes were washed 3× 15 min with PBS-T and treated with HRP-coupled secondary antibodies (1:7000 in 1.25% BSA or milk in PBS-T) for 1 hour at RT. HRP was detected by enhanced chemiluminescence using the LumiGlo reagent kit (Cell Signaling Technologies) according to the manufacturer's specifications and visualized on ECL Amersham Hyperfilms (GE Healthcare). Images were cropped for presentation. WB antibodies used: anti-MALT1 (B-12 for human, D-1 for murine), anti-β-Actin (C4, 1:20.000), anti-CYLD (E-10), anti-HOIL-1 (H-1), (all Santa Cruz); anti-Regnase-1 (MAB7875) (R&D); anti-TRAF6 (EP591Y) (Abcam); anti-Roquin-1/2 (clone 3F12, 1:10, HMGU core facility monoclonal antibodies); horseradish peroxidase (HRP)-conjugated secondary antibodies (Jackson ImmunoResearch, 1:7000); all antibodies were used at 1:1000 dilution if not otherwise stated.

Example 14—Loss of MALT1-TRAF6 interaction by single missense mutations increases MALT1 protease activity in resting T cells. To address a T cell-intrinsic impact of TRAF6 on MALT1 protease activity in the absence of secondary effects caused by the autoimmune/inflammatory phenotype, the inventors switched to a heterologous system. The inventors transduced MALT1 KO Jurkat T cells with MALT1A and MALT1B WT and the respective TRAF6 binding mutants (human MALT1A E316A/E806A and MALT1B E795A); which abrogates interaction of MALT1 and TRAF6. As previously described, MALT1A E316A/E806A and MALT1B E795A failed to rescue I_{K}Bα degradation and thus NF-_{K}B activation after P/I stimulation (I. Meininger et al., Alternative splicing of MALT1 controls signalling and activation of CD4(+) T cells. Nature communications 7, 11292 (2016)). As observed in Malt1 TBM-T CD4+ T cells, constitutive cleavage of MALT1 substrates CYLD, Regnase-1 and HOIL-1 was detected in MALT1 KO Jurkat T cells rescued with MALT1A E316A/E806A or MALT1B E795A, but not the respective WT isoforms (FIG. 2B). The inventors used a biotinylated MALT1 activity-based probe (bio-MALT1-ABP), which covalently binds active MALT1 and allows direct measurement of cellular MALT1 protease activity (A. C. Eitelhuber et al., Activity-based probes for detection of active MALT1 paracaspase in immune cells and lymphomas. Chemistry & biology 22, 129-138 (2015)). Increased activity of MALT1 was detected in resting Jurkat T cells containing the TRAF6 binding mutants MALT1A E316A/E806A (˜50 fold) or MALT1B E795A (˜5 fold) (FIG. 2B). Importantly, constitutive protease activity detected by MALT1 ABP or substrate cleavage was abolished in the MALT1B active site mutant C453A, proving that it relies directly on constitutive protease activity upon loss of TRAF6 interaction (FIG. 2B). The inventors recently identified the human hypomorphic germline MALT1 mutation (c.2418G>C) which leads to the Glu(E) to Asp(D) exchange in the T6BM2 motif (N. Kutukculer et al., Human immune disorder associated with homozygous hypomorphic mutation affecting MALT1B splice variant. J Allergy Clin Immunol 147, 775-778 e778 (2021)). The missense mutation selectively affects TRAF6 binding and NF-_{K}B activation of the MALT1B isoform, which causes a severe primary immune disorder with signs of immune deficiency and autoimmunity. Indeed, the conserved E795D mutation in MALT1B does not only abrogate NF-_{K}B signaling, but it also induces constitutive MALT1B activation comparable to MALT1B E795A exchange as revealed by the increased cleavage of MALT1 substrates CYLD, Regnase-1 and HOIL-1 (FIG. 2C). Thus, a single human germline mutation in the second TRAF6 binding motif (T6BM2) is sufficient to render MALT1B proteolytic active.

Example 15—Deletion of TRAF6 and mutation of TRAF6 catalytic activity or TRAF6 dimerization increases MALT1 protease activity in resting T cells. The inventors asked if and how TRAF6 negatively impacts MALT1 protease activity in Jurkat T cells. The inventors generated TRAF6 KO Jurkat T cell clones using CRISPR/Cas9 technology. As previously observed, NF-_{K}B signaling and transcriptional activity was impaired in TRAF6 KO Jurkat T cells after TCR/CD28 or P/I stimulation, verifying the essential role of TRAF6 for TCR-induced NF-_{K}B activation. However, MALT1 protease was constitutively active as evident from augmented MALT1 activity as detected by MALT1 activity-based probes (3-4 fold increase compared to Jurkat T cells with TRAF6) (FIG. 3A). The inventors reconstituted TRAF6 KO Jurkat T cells with TRAF6 WT, TRAF6 C70A E3 ligase mutant that cannot bind to UBC13/UEV1A E2 enzyme or the TRAF6 R88A/F118A oligomerization mutant (Q. Yin et al., E2 interaction and dimerization in the crystal structure of TRAF6. Nature structural & molecular biology 16, 658-666 (2009)). TRAF6 WT rescued counteracted chronic MALT1 protease activity as seen by diminished cleavage of substrate CYLD and

Regnase-1 in the absence of stimulation (FIG. 3B). Neither TRAF6 C70A nor TRAF6 R88A/F118A mutants could compensate for the loss or TRAF6, demonstrating that TRAF6 E3 ligase activity and self-assembly is required to retain MALT1 protease inactive in resting T cells (FIG. 3B, C). Thus, just like loss of MALT1-TRAF6 interaction, inactivation of TRAF6 by ablation, mutation of activity or mutation of oligomerization renders MALT1 protease constitutively active.

Example 16—Binding of MALT1 to TRAF6 and TRAF6-dependent control ofMALT1 protease activity in CD4 T cells is critical for T cell homeostasis. To investigate the T cell-intrinsic role of MALT1-TRAF6 interaction and TRAF6 expression in the regulation of MALT1 protease and maintenance of immune homeostasis, the inventors generated Malt1^{TBM/ﬂ};CD4-Cre (Malt1 TBM-T) mice. Malt1 TBM (TRAF6 binding mutant) mice contain destructive mutations in the two TRAF6 binding motifs (T6BM), rendering MALT1A and MALT1B unable to interact with TRAF6 (I. Meininger et al., Alternative splicing of MALT1 controls signalling and activation of CD4(+) T cells. Nature communications 7, 11292 (2016)). The inventors used CRISPR/Cas9 editing to introduce missense mutations in the Malt 1 gene in murine embryonic stem (ES) cells yielding Malt1 TRAF6 binding mutant (TBM) mice with loss-of-function mutations in T6BM1/2 of MALT1A (E325A;E814A) and T6BM2 of MALT1B (E803A) (FIG. 6A). ES cells containing homozygous alterations in both T6BMs were injected into embryos of C57BL/6 mice. Chimeric offspring containing the mutant allele were crossed to C57BL/6 mice to obtain heterozygous MALT1^{TBM/+} mice, which were further crossed for obtaining homozygous Malt1^{TBM/TBM} (Malt1 TBM) mice. Differential PCR and genomic sequencing of Malt1 WT and TBM alleles verified correct genome editing and Western Blot showed equivalent protein expression of MALT1 in T cells of Malt1^{+/+}, Malt 1^{TBM/+} and Malt1^{TBM/TBM} mice. The inventors generated Malt1^{TBM/ﬂ}; CD4-Cre (Malt1 TBM-T) mice by crossing to Malt1 floxed mice and thus conditional deletion of one floxed Malt1 allele in T cells expressing CD4-Cre. Also, Traf6^{ﬂ/ﬂ}; CD4-Cre (Traf6-ΔT) mice were bred, which lack TRAF6 expression in CD4+ and CD8+ T cells (C. G. King, et al., TRAF6 is a T cell-intrinsic negative regulator required for the maintenance of immune homeostasis. Nat Med 12, 1088-1092 (2006)). Surprisingly, the inventors consistently noticed augmented cleavage of MALT1 substrates CYLD and Regnase-1 in isolated CD4+ T cells purified from Malt1 TBM-T and Traf6-ΔT mice. Substrate cleavage was visible even in the absence of T cell stimulation, indicating constitutive MALT1 protease activation as evident from CYLD, HOIL-1, Regnase-1, Roquin1 and Roquin2 substrate cleavage upon loss of TRAF6 expression or destruction of MALT1-TRAF6 interaction (FIGS. 4A and B). To determine functional consequences, the inventors analyzed T cell activation by measuring the number of CD44^{hi}CD62^{lo} CD4+ and CD8+ T effector memory (T_{EM}) cells in Malt1 TBM-T and Traf6-ΔT mice (FIG. 4C). Indeed, in both cases T cells become activated through a cell-autonomous and cell-intrinsic mechanism upon destruction of MALT1-TRAF6 interaction or TRAF6 ablation. Further, the inventors analyzed expression of NFKBID/I_{K}BNS and ICOS in Malt1 TBM-T and Traf6-ΔT CD4+ T cells, which are tightly controlled by the post-transcriptional regulators and MALT1 substrates Regnase-1 and Roquin-1/2 (A. Gewies et al., Uncoupling Malt1 threshold function from paracaspase activity results in destructive autoimmune inflammation. Cell reports 9, 1292-1305 (2014), N. Rehage et al., Binding of NUFIP2 to Roquin promotes recognition and regulation of ICOS mRNA. Nature communications 9, 299 (2018), T. Uehata et al., Malt1-induced cleavage of regnase-1 in CD4(+) helper T cells regulates immune activation. Cell 153, 1036-1049 (2013), K. Essig et al., Roquin targets mRNAs in a 3′-UTR-specific manner by different modes of regulation. Nature communications 9, 3810 (2018)). In agreement with augmented cleavage of Regnase-1 and Roquin-1/2, I_{K}BNS and ICOS expression was upregulated in CD4+ and CD8+ T cells from Malt1 TBM-T and Traf6-ΔT mice (FIGS. 4D and E). Thus, these data provide evidence that complete loss of TRAF6 or selective loss of MALT1-TRAF6 interaction leads to deregulated constitutive MALT1 protease activation in T cells, which is sufficient to partially inactivate post-transcriptional control mechanisms by the RNA binding proteins (RBPs) Regnase-1 and Roquin-1/2, leading to enhanced I_{K}BNS and ICOS expression and cell autonomous T cell activation.

Example 17—MALT1 inhibitor treatment inhibits substrate cleavage and restores T effector cell homeostasis after loss of TRAF6. To prove that constitutive MALT1 protease activation is the cause for T cell activation and differentiation into T_{EM} cells in Traf6-ΔT mice, the inventors treated the mice with the potent MALT1 inhibitor MLT-985 (J. Quancard et al., Optimization of the In Vivo Potency of Pyrazolopyrimidine MALT1 Protease Inhibitors by Reducing Metabolism and Increasing Potency in Whole Blood. J Med Chem 63, 14594-14608 (2020)). The inventors started therapeutic treatment of Traf6-ΔT mice with MLT-985 at 8 weeks of age after the onset of T cell hyper-activation. MLT-985 was administered at 16 mg/kg (i.p., BID) for 10 consecutive days to achieve optimal MALT1 inhibition throughout the treatment (FIG. 5A). At the end of the treatment period, splenic CD4+ T cells were isolated and Western blot demonstrated that MLT-985 treatment effectively abolished constitutive MALT1 cleavage of the substrates CYLD, Regnase-1 and HOIL1 in Traf6-ΔT mice when compared to vehicle control (FIG. 5B). Immune phenotyping demonstrated that concomitant T cell activation was ameliorated in Traf6-ΔT mice after 10 days of MALT1 inhibitor treatment, as reflected by a strong decrease in relative numbers of T_{EM} CD4+ and CD8+ T cells (FIG. 5C). Further, MALT1 activity markers I_{K}BNS and ICOS were reduced in expression after MLT-985 treatment (FIG. 5D). In fact, T cell activation markers and MALT1 activity markers I_{K}BNS and ICOS were down to the levels detected in healthy WT mice. Thus, T cell hyper-activation in Traf6-ΔT mice is completely reverted by MALT1 inhibition, providing evidence that loss of TRAF6 drives autoimmune inflammation by unopposed MALT1 protease activity in T_{EM} cells.

Example 18—MALT1 protease drives inflammation upon loss of MALT1-TRAF6 binding. The inventors wanted to prove that MALT1 protease activation is the cause for T cell activation after loss of MALT1-TRAF6 interaction. Thus, the inventors compared phenotypes of Malt1^{TBM/TBM} (Malt1 TBM) mice, containing inactivating missense mutations in the two T6BMs of MALT1, and Malt1^{TBMPM/TBMPM} mice, which in addition contained a missense mutation that renders MALT1 protease inactive (PM: Paracaspase mutant) (FIG. 6A). As previously described, Malt1 TBM mice were generated targeting murine ES cells with CRISPR/Cas9. The inventors introduced the PM C472A by CRISPR/Cas9 and homology directed repair in Malt1^{TBM/+} zygotes. The approach yielded offspring with the correct MALT1 paracaspase mutation (PM) on the same allele as the T6BM1/2 mutations and the inventors ultimately obtained homozygous Malt1^{TBMPM/TBMPM} (Malt1 TBMPM) mice. Malt1 TBM mice stopped thriving between 3 to 4 weeks of age, showed a hunched posture and had to be euthanized between 3-6 weeks after birth at a median age of 27 days (FIG. 6B). In contrast to homozygous Malt1^{TBM/TBM} mice, Malt1^{TBMPM/TBMPM} mice appeared healthy, and no weight loss, enlarged spleens or macroscopic signs of inflammation were observed 8 weeks after birth. In contrast to Malt1 TBM-T mice (FIG. 4A), no constitutive cleavage of MALT1 substrate CYLD, Regnase-1 and HOIL-1 was observed in CD4+ T cells from Malt1^{TBMPM/TBMPM} mice and MALT1 activity was also not inducible after P/I stimulation (FIG. 6C). The inventors investigated the T cell populations in Malt1 TBM and Malt1 TBMPM mice. Inspection of T cells demonstrated a massive accumulation of CD44^{hi}CD62L^{lo} CD4+ and CD8+ effector/memory T cells (T_{EM}) in spleen and lymph nodes of Malt1 TBM mice (FIG. 6D). Contrary to immune activation observed in Malt1 TBM mice, there was even a decrease in CD44^{hi}CD62L^{lo} CD4+ and CD8+ T_{EM} cells in Malt1^{TBMPM/TBMPM} mice compared to heterozygous littermate controls (FIG. 6E). An autoimmune pathology upon defective protease activity in Malt1 PM mice has been attributed to a strong decrease in regulatory T (Treg) cell numbers and function (F. Bornancin et al., Deficiency of MALT1 paracaspase activity results in unbalanced regulatory and effector T and B cell responses leading to multiorgan inflammation. Journal of immunology (Baltimore, Md.: 1950) 194, 3723-3734 (2015), A. Gewies et al., Uncoupling Malt1 threshold function from paracaspase activity results in destructive autoimmune inflammation. Cell reports 9, 1292-1305 (2014), M. Jaworski et al., Malt1 protease inactivation efficiently dampens immune responses but causes spontaneous autoimmunity. The EMBO journal 33, 2765-2781 (2014)). In contrast, numbers of CD4+ FoxP3+ Treg cells were increased in Malt1 TBM mice compared to heterozygous littermates (FIG. 6F). As observed in Malt1^{-/-} mice, Malt1^{TBMPM/TBMPM} mice are nearly devoid of peripheral Treg cells (FIG. 6G). Further, eTreg cells from Malt1 TBM homo- and heterozygous mice expressed even higher levels of the suppression markers OX40 (FIG. 6H). As a result of the severely increased T_{EM} cell population, Malt1 TBM mice displayed auto-inflammation and upregulation of cytokines TNFα, IFNγ, IL-6, IL-10 and IL-17 (FIG. 6I). In contrast, the most strongly upregulated cytokines TNFα and IFNg remained low in the serum of Malt1^{TBMPM/TBMPM} mice (FIG. 6J). Taken together, loss of TRAF6 binding to MALT1 leads to spontaneous lymphocyte activation and an immune pathology, which is not driven by functional loss of Treg cells, but increase in the T_{EM} cells. All of the symptoms and markers are completely relying on MALT1 protease activity, proving that constitutive MALT1 leads to a strong T effector cell activation that is not counteracted by Treg cells.

Example 19—The patient derived E->D mutation of MALT1 TBM2 is sufficient to cause T effector memory responses. The recently identified human hypomorphic germline MALT1 mutation (c.2418G>C) which leads to the Glu(E) to Asp(D) exchange in the T6BM2 motif, also renders MALT1B constitutively active as a result of lost MALT1 binding (N. Kutukculer et al., Human immune disorder associated with homozygous hypomorphic mutation affecting MALT1B splice variant. J Allergy Clin Immunol 147, 775-778 e778 (2021)) (FIG. 2B). Since the mutation causes a severe immune syndrome in humans, with similar signs of autoimmunity seen in Malt1 TBM mice, the inventors explored the intriguing possibility that this single missense mutation in the murine Malt1 gene may cause T cell activation. The inventors introduced the TBM2 E814D exchange by CRISPR/Cas9 and homology directed repair in zygotes of mice (FIG. 7A). Indeed, analyses of Malt1TBM2/TBM2 offspring showed that this single mutation was causing a strong increase in CD44^{hi}CD62L^{lo} CD4+ and CD8+ T_{EM} cells in mice, proving that a destruction of TRAF6 binding motif 2 is sufficient to cause T cell activation (FIG. 7B).

Example 20—Malt1 TBM mutations result in an enrichment of CD8+ cytotoxic T cells in the tumor environment. Adoptive T cell therapy is a method by which patient T cells are modified to yield improved cytotoxic, anti-tumor activity. To prove that the Malt1 TBM mutations provide a benefit for T cells in adoptive T cell therapy, Malt1 TBM-T mice were bred to have additional expression of a transgenic OT1+ T cell receptor which recognizes an Ovalbumin (Ova) peptide presented in MHC class I complexes on the surface of B16-Ova murine melanoma cells. CD8+ T cells from donor mice with and without Malt1 TBM mutations were transferred via tail vein injection into mice with subcutaneous B16-Ova tumors (FIG. 8A). Injected T cells express the congenic marker CD45.2 on the cell surface, allowing them to be differentiated from endogenous T cells in the receptor mice, which express exclusively CD45.1. Mice were sacrificed after 3 weeks and tumor, spleen and axillary lymph nodes were examined for donor-derived OT1+ T cells expressing donor-derived CD45.2 cells (FIG. 8B). Relative numbers of donor-derived T cells carrying the Malt1 TBM mutations were significantly enriched in the tumor tissue on average by approximately 40-fold compared to mice which received Malt1 wildtype T cells. This enrichment was not seen in the peripheral lymphoid organs spleen or axillary lymph nodes (FIG. 8B). Malt1 TBM mutations provide a benefit to OT1+ T cells for use in adoptive T cell therapy. Malt1 TBM mutations in combination with a tumor neo-antigen, such as the Ova-peptide on the B16 cells, provokes a strong and selective enrichment of cytotoxic CD8 T cells in the vicinity of the tumor, without causing an overall increase in the number of peripheral CD8 T cells. In the context of adoptive T cell transfer therapies, the tumor enrichment of T cells carrying the Malt1 TBM mutations or other changes that render MALT1 constitutively active will yield a distinct advantage in attacking the cancer cells either alone or when combined with immune activating stimuli/adjuvants. The immune activating stimuli may be an innate immune stimulus such as CpG oligodeoxynucleotides (CPG ODN) or any other innate, adaptive or adjuvant immune trigger (FIG. 8C). The immune trigger may be a biologic or chemical agent, such as a protein (e.g. antigenic peptides, cytokine, therapeutic antibody etc.), a nucleic acid (e.g. mRNA vaccine, oligonucleotides, etc.), an inorganic or organic immune adjuvant (e.g. aluminium, Freund's adjuvant, bacterial products, paraffin oils etc.) or any other chemical compound that modulates the immune response and enhances cytotoxic killing of tumor cells by T cells carrying the MALT1 activating mutations.

Unless otherwise stated, the following terms used in this document, including the description and claims, have the definitions given below.

Those skilled in the art will recognize, or be able to ascertain, using not more than routine experimentation, many equivalents to the specific embodiments of the invention described herein. Such equivalents are intended to be encompassed by the present invention.

It is to be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only and is not intended to limit the scope of the present invention, which is defined solely by the claims.

All publications cited throughout the text of this specification (including all patents, patent applications, scientific publications, manufacturer's specifications, instructions, etc.) are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

1. Cell of the human immune system transduced or transfected with a T cell receptor (TCR) or chimeric antigen receptor (CAR), said cell being further modified to render its MALT1 protease activity constitutive active.

2. The cell of claim 1, wherein said cell of the human immune system is a T cell, NK cell, NKT cell, a B cell or a macrophage, preferably wherein said T cell is a CD8+ T cell, a CD4+ T cell or a Treg cell.

3. The cell of claim 2, wherein the constitutive active MALT1 protease activity of said cell is characterized by increased MALT1 protease activity in comparison to MALT1 protease activity of a cell of the immune system transduced or transfected with a T cell receptor (TCR) or chimeric antigen receptor (CAR), but which is not further modified to render its MALT1 protease activity constitutive active, preferably wherein MALT1 protease activity is determined by cleavage of MALT1 substrates such as Regnase-1, Roquin1, Roquin2, N4BP1, HOIL-1/RBCK1, CYLD, A20/TNFAIP3, BCL10, MALT1 or RelB, preferably wherein MALT1 protease activity is determined by cellular activity-based assay utilizing a MALT1 activity-based probe (ABP) such as Biotin-LVSR-AOMK, Biotin-LRSR-AOMK or BODIPY-LVPipR-AOMK.

4. The cell of any one of the preceding claims, wherein MALT1 protease activity is rendered constitutive active by abolishing MALT1 interaction with TRAF6 or by deletion of TRAF6, preferably, wherein MALT1 interaction with TRAF6 is abolished by inactivating the TRAF6 binding motif 1 (T6BM1) of human MALT1A and/or the TRAF6 binding motif 2 (T6BM2) of human MALT1A or human MALT1B, preferably, wherein T6BM1 encompasses amino acids 314 to 319 of human MALT1A shown in SEQ ID NO: 1 and T6BM2 encompasses amino acids 804 to 809 of human MALT1A shown in SEQ ID NO: 1 or amino acids 793 to 798 of human MALT1B shown in SEQ ID NO: 2.

5. The cell of claim 4, wherein MALT1 protease activity is rendered constitutive active by substituting E for another amino acid, preferably E→A or E→D at a position in a MALT1A protein corresponding to position 316 in SEQ ID NO: 1, or wherein MALT1 protease activity is rendered constitutive active by substituting E for another amino acid, preferably E→A or E→D at a position in a MALT1A protein corresponding to position 806 of the MALT1A protein shown in SEQ ID NO: 1 or at a position in a MALT1B protein corresponding to position to position 795 of the MALT1B protein shown in SEQ ID NO: 2, or wherein MALT1 protease activity is rendered constitutive active by
(i) substituting Y→A at a position in a MALT1A protein corresponding to position 657 of the MALT1A protein shown in SEQ ID NO: 1;
(ii) substituting an amino acid at a position in a MALT1A protein corresponding to position 506 of the MALT1A protein shown in SEQ ID NO: 1, wherein the amino acid substitution is selected from the group consisting of L→A, L→G, and L→K,
(iii) substituting N→A at a position in a MALT1A protein corresponding to position N508 of the MALT1A protein shown in SEQ ID NO: 1,
(iv) substituting Y→A at a position in a MALT1A protein corresponding to position Y367 of the MALT1A protein shown in SEQ ID NO: 1, or wherein MALT1 protease activity is rendered constitutive active by
(v) substituting Y→A at a position in a MALT1B protein corresponding to position 646 of the MALT1B protein shown in SEQ ID NO: 2,
(vi) substituting Y→A at a position in a MALT1B protein corresponding to position L495 of the MALT1B protein shown in SEQ ID NO:2,
(vii) substituting Y→A at a position in a MALT1B protein corresponding to position N495 of the MALT1B protein shown in SEQ ID NO:2, or
(viii) substituting Y→A at a position in a MALT1B protein corresponding to position Y356 of the MALT1B protein shown in SEQ ID NO:2.

6. The cell of any one of the preceding claims, wherein MALT1 protease activity is rendered constitutive active by inactivating TRAF6 through inactivating TRAF6 gene expression, rendering TRAF6 E3 ligase inactive or abolishing TRAF6 oligomerization, preferably wherein MALT1 protease activity is rendered constitutive active by
(i) inactivating the C-terminal MATH domain corresponding to position 350-499 of the TRAF6 protein shown in SEQ ID NO: 3,
(ii) substituting D→K at a position in a TRAF6 protein corresponding to position 57 of the TRAF6 protein shown in SEQ ID NO:3,
(iii) substituting C→A at a position in a TRAF6 protein corresponding to position 70 of the TRAF6 protein shown in SEQ ID NO: 3,
(iv) substituting an amino acid at a position in a TRAF6 protein corresponding to position 72 of the TRAF6 protein shown in SEQ ID NO:3, wherein the amino acid substitution is selected from the group consisting of I→D, I→A, I→K, and I→F,
(v) substituting an amino acid at a position in a TRAF6 protein corresponding to position 74 of the TRAF6 protein shown in SEQ ID NO: 3, wherein the amino acid substitution is selected from the group consisting of L→H, L→E, L→K, and L→R, or
(vi) substituting R→A at a position in a TRAF6 protein corresponding to position 88 of the TRAF6 protein shown in SEQ ID NO: 3 and substituting F→A at a position in a TRAF6 protein corresponding to position 118 of the TRAF6 protein shown in SEQ ID NO: 3.

7. The cell of any one of the preceding claims, wherein MALT1 protease activity is rendered constitutive active by inactivating a protein essential for the regulation of TRAF6 E3 ligase activity, such as the E2 enzymes UBC13 and UEV1A.

8. The cell of any one of the preceding claims, wherein MALT1 substrates are genetically inactivated.

9. The cell of any one of the preceding claims, wherein said cell is additionally modified to comprise a suicide gene.

10. Cell of any one of claims 1 to 9 for use as a medicament.

11. Cell of any one of claims 1 to 9 for use in adoptive T cell therapy.

12. Cell of any one of claims 1 to 9 for use in a method of treating cancer.

13. Cell of any one of claims 1 to 9 for use in a method of enriching the cell at a target site in a subject.

14. The cell for use of claim 13, wherein the method further comprises providing an agent for immune system stimulation.

15. A method for generating a cell of any one of claims 1 to 9, comprising modifying a cell to render MALT1 protease activity constitutive active, preferably wherein said modifying step is preceded by the step transducing or transfecting a T cell, NK cell, NKT cell or a macrophage with a heterologous TCR or CAR.

16. An in vitro method of enhancing the activity of a cell of the human immune system transduced or transfected with a T cell receptor (TCR) or chimeric antigen receptor (CAR), comprising modifying said cell in that MALT1 is rendered constitutive active, preferably wherein said cell of the immune system is a T cell, NK cell, NKT cell, or a macrophage, preferably wherein activity of a cell of the human immune system is monitored by increased activation markers, such as T cell activation markers and/or increased cytokine production.

17. In vitro use of constitutive active MALT1 for enhancing the activity of a cell transduced or transfected with a T cell receptor (TCR) or chimeric antigen receptor (CAR).


SEQUENCE LISTING (3 sequences)

SEQ ID NO: 1 - protein, 824 aa, homo sapiens
  MALT1A
MSLLGDPLQALPPSAAPTGPLLAPPAGATLNRLREPLLRRLSELLDQAPEGRGWRRLAELAGSRGRLRLSCLDLEQCSLKVLEPEGSPSLCLLKLMGEKGCTVTELSDFLQAMEHTEVLQLLSPPGIKITVNPESKAVLAGQFVKLCCRATGHPFVQYQWFKMNKEIPNGNTSELIFNAVHVKDAGFYVCRVNNNFTFEFSQWSQLDVCDIPESFQRSVDGVSESKLQICVEPTSQKLMPGSTLVLQCVAVGSPIPHYQWFKNELPLTHETKKLYMVPYVDLEHQGTYWCHVYNDRDSQDSKKVEIIIGRTDEAVECTEDELNNLGHPDNKEQTTDQPLAKDKVALLIGNMNYREHPKLKAPLVDVYELTNLLRQLDFKVVSLLDLTEYEMRNAVDEFLLLLDKGVYGLLYYAGHGYENFGNSFMVPVDAPNPYRSENCLCVQNILKLMQEKETGLNVFLLDMCRKRNDYDDTIPILDALKVTANIVFGYATCQGAEAFEIQHSGLANGIFMKFLKDRLLEDKKITVLLDEVAEDMGKCHLTKGKQALEIRSSLSEKRALTDPIQGTEYSAESLVRNLQWAKAHELPESMCLKFDCGVQIQLGFAAEFSNVMIIYTSIVYKPPEIIMCDAYVTDFPLDLDIDPKDANKGTPEETGSYLVSKDLPKHCLYTRLSSLQKLKEHLVFTVCLSYQYSGLEDTVEDKQEVNVGKPLIAKLDMHRGLGRKTCFQTCLMSNGPYQSSAATSGGAGHYHSLQDPFHGVYHSHPGNPSNVTPADSCHCSRTPDAFISSFAHHASCHFSRSNVPVETTDEIPFSFSDRLRISEK

SEQ ID NO: 2 - protein, 813 aa, homo sapiens
  MALT1B
MSLLGDPLQALPPSAAPTGPLLAPPAGATLNRLREPLLRRLSELLDQAPEGRGWRRLAELAGSRGRLRLSCLDLEQCSLKVLEPEGSPSLCLLKLMGEKGCTVTELSDFLQAMEHTEVLQLLSPPGIKITVNPESKAVLAGQFVKLCCRATGHPFVQYQWFKMNKEIPNGNTSELIFNAVHVKDAGFYVCRVNNNFTFEFSQWSQLDVCDIPESFQRSVDGVSESKLQICVEPTSQKLMPGSTLVLQCVAVGSPIPHYQWFKNELPLTHETKKLYMVPYVDLEHQGTYWCHVYNDRDSQDSKKVEIIIDELNNLGHPDNKEQTTDQPLAKDKVALLIGNMNYREHPKLKAPLVDVYELTNLLRQLDFKVVSLLDLTEYEMRNAVDEFLLLLDKGVYGLLYYAGHGYENFGNSFMVPVDAPNPYRSENCLCVQNILKLMQEKETGLNVFLLDMCRKRNDYDDTIPILDALKVTANIVFGYATCQGAEAFEIQHSGLANGIFMKFLKDRLLEDKKITVLLDEVAEDMGKCHLTKGKQALEIRSSLSEKRALTDPIQGTEYSAESLVRNLQWAKAHELPESMCLKFDCGVQIQLGFAAEFSNVMIIYTSIVYKPPEIIMCDAYVTDFPLDLDIDPKDANKGTPEETGSYLVSKDLPKHCLYTRLSSLQKLKEHLVFTVCLSYQYSGLEDTVEDKQEVNVGKPLIAKLDMHRGLGRKTCFQTCLMSNGPYQSSAATSGGAGHYHSLQDPFHGVYHSHPGNPSNVTPADSCHCSRTPDAFISSFAHHASCHFSRSNVPVETTDEIPFSFSDRLRISEK

SEQ ID NO: 3 - protein, 522 aa, homo sapiens
  TRAF6
MSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAQAVHSLSVIPDSGYISEVRNFQETIHQLEGRLVRQDHQIRELTAKMETQSMYVSELKRTIRTLEDKVAEIEAQQCNGIYIWKIGNFGMHLKCQEEEKPVVIHSPGFYTGKPGYKLCMRLHLQLPTAQRCANYISLFVHTMQGEYDSHLPWPFQGTIRLTILDQSEAPVRQNHEEIMDAKPELLAFQRPTIPRNPKGFGYVTFMHLEALRQRTFIKDDTLLVRCEVSTRFDMGSLRREGFQPRSTDAGV